(19) United States
(12) Patent Application Publication
(10) Pub. No.: US 2025/0342964 A1
(43) Pub. Date: Nov. 6, 2025

(54) Cancer Classification with Tissue of Origin Thresholding

(71) Applicant: Grail, Inc., Menlo Park, CA (US)

(72) Inventors: Qinwen Liu, Fremont, CA (US); Oliver Claude Venn, San Francisco, CA (US); Samuel S. Gross, Sunnyvale, CA (US); Robert Abe Paine Calef, Redwood City, CA (US)

(21) Appl. No.: 19/265,345

(22) Filed: Jul. 10, 2025

Related U.S. Application Data
(63) Continuation-in-part of application No. 17/066,863, filed on Oct. 9, 2020, now abandoned.
(60) Provisional application No. 62/914,341, filed on Oct. 11, 2019.
(60) Provisional application No. 63/024,033, filed on May 13, 2020.
(60) Provisional application No. 63/041,699, filed on Jun. 19, 2020.

(51) Int. Cl. G16H 50/20; G06N 20/00; G16B 20/20; G16B 40/10; G16B 40/20; G16H 20/10; G16H 50/30; G16H 50/70

(52) U.S. Cl. CPC G16H 50/20; G06N 20/00; G16B 20/20; G16B 40/10; G16B 40/20; G16H 20/10; G16H 50/30; G16H 50/70

(57) ABSTRACT

Methods and systems for detecting cancer and/or determining a cancer tissue of origin are disclosed. In some embodiments, a multiclass cancer classifier is disclosed that is trained with a plurality of biological samples containing cfDNA fragments. The analytics system derives a feature vector for each sample, and the multiclass classifier predicts a probability likelihood for each of a plurality of tissue of origin (TOO) classes. In some embodiments, the plurality of TOO classes include hematological subtypes, including both hematological malignancies and precursor conditions. In one embodiment, non-cancer samples having high tissue signal are pruned from the training sample set. In another embodiment, the analytics system stratifies samples according to tissue signal and applies binary threshold cutoffs determined for each stratum.

### CROSS REFERENCE TO RELATED APPLICATIONS

This application is a continuation-in-part of U.S. application Ser. No. 17/066,863, filed Oct. 9, 2020, which claims the benefit of U.S. Provisional Application No. 63/041,699, filed Jun. 19, 2020, and U.S. Provisional Application No. 63/024,033, filed May 13, 2020, and U.S. Provisional Application No. 62/914,341, filed Oct. 11, 2019, all of which are incorporated by reference in its their entirety.

### BACKGROUND

### Field of Art

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. However, there remains a need in the art for improved methods for analyzing methylation sequencing data from cell-free DNA for the detection, diagnosis, and/or monitoring of diseases, such as cancer.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of somatic variant, methylation status, or other genetic aberrations) from a blood sample can provide insight into whether a subject may have cancer, and further insight on what type of cancer the subject may have. Towards that end, this description includes systems and methods for analyzing cell-free DNA sequencing data for determining a subject's likelihood of having a disease.

An analytics system processes a multitude of sequencing data from a plurality of samples (e.g., a plurality of cancer and non-cancer samples) to identify features that are subsequently utilized for cancer classification. With the sequencing data, the analytics system is able to train and deploy a cancer classifier for generating a cancer prediction for a test sample.

Regarding which training samples are used to train the cancer classifier, the analytics uses training samples that have already been identified and labeled as having one or a number of cancer types, as well as training samples that are from healthy individuals that are labeled as non-cancer. Each training sample includes a set of fragments. For each training sample, the analytics system generates a feature vector, for example, by assigning a score to each of the identified features. The analytics system may group the training samples into sets of one or more training samples for iterative training of the cancer classifier. The analytics system inputs each set of feature vectors into the cancer classifier and adjusts classification parameters in the cancer classifier such that a function of the cancer classifier calculates cancer predictions that accurately predict the labels of the training samples in the set based on the feature vectors and the classification parameters. After iterating the above steps through each set of training samples, the cancer classifier is sufficiently trained.

During deployment, the analytics system generates a feature vector for a test sample in a similar manner to the training samples, e.g., by assigning a score to each of a plurality of features in a feature vector for each of the test samples. Then the analytics system inputs the feature vector for the test sample into the cancer classifier which returns a cancer prediction. In one embodiment, the cancer classifier may be configured as a binary classifier to return a cancer prediction of a likelihood of having or not having cancer. In another embodiment, the cancer classifier may be configured as a multiclass classifier to return a cancer prediction with prediction values for the cancer types being categorized.

The present disclosure provides methods and systems for detecting cancer and/or determining a cancer tissue of origin. In some embodiments, the invention comprises a method, or system, for detecting cancer, comprising: receiving sequencing data for a plurality of biological samples containing cfDNA fragments, the biological samples comprising cancer and non-cancer samples; for each non-cancer sample of the plurality of biological samples: classifying the biological sample using a multiclass classifier based on features derived from the sequencing data, wherein the multiclass classifier predicts a probability likelihood for each of a plurality of tissue of origin classes, the plurality of tissue of origin classes further comprising one or more tissue of origin subtype classes; and determining, for each subtype class, whether the predicted probability likelihood exceeds a subtype cutpoint, wherein the subtype cutpoint is indicative of a specificity threshold for the subtype class; and determining a threshold cutoff for predicting a presence or absence of cancer, the threshold cutoff determined based on a distribution of probability scores corresponding to the non-cancer samples, wherein the distribution of probability scores excludes probability scores associated with one or more non-cancer samples identified as having a probability likelihood that exceeds a subtype cutpoint.

In some embodiments, the distribution of probability scores is generated by a binary classifier trained on training samples derived from the cancer and non-cancer samples.

In some embodiments, the training samples are divided into multiple cross-validation training sets and used to train the binary classifier for detecting the presence of cancer, wherein the binary classifier produces, for each training sample, a probability score indicating a presence or absence of cancer.

In some embodiments, the binary classifier is associated with a first threshold cutoff, and wherein determining the threshold cutoff for predicting a presence or absence of cancer comprises modifying the first threshold cutoff based on excluding the probability scores associated with the one or more non-cancer samples identified as having a probability likelihood that exceeds a subtype cutpoint.

In some embodiments, the threshold cutoff comprises applying a desired specificity level to the distribution of probability scores, the threshold cutoff comprising a threshold probability score.

In some embodiments, the method or system comprises receiving test sequencing data for a test biological sample containing cfDNA fragments; analyzing the test sequencing data to determine a test probability score for a presence or absence of cancer; determining whether the test probability score exceeds the threshold cutoff; and in response to determining that the test probability score exceeds the threshold cutoff, predicting a presence of cancer.

In some embodiments, the method or system further comprises in response to determining that the test probability score does not exceed the threshold cutoff, predicting an absence of cancer.

In some embodiments, the method or system further comprises in response to determining that the test probability score exceeds the threshold cutoff, assessing the test sequencing data for a tissue of origin of the cancer using the multiclass classifier.

In some embodiments, the multiclass classifier is trained on training samples derived from the cancer and non-cancer samples.

In some embodiments, the method or system further comprises determining each subtype cutpoint by an iterative optimization process that optimizes tradeoff between a clinical specificity and a clinical sensitivity for the corresponding tissue of origin subtype class.

In some embodiments, the tissue of origin subtype classes comprise hematological classes indicative of one or more hematological conditions. In some embodiments, each subtype cutpoint for each hematological class is determined based on a measure of clinical aggressiveness of the corresponding hematological condition.

In some embodiments, the measure of clinical aggressiveness comprises one or more of: early phase of disease progression, survival rate, speed of disease progression, and severity of the disease.

In some embodiments, the hematological classes comprise a NHL_indolent class, a myeloid class, and a circulating_lymphoid class. In some embodiments, the hematological classes comprise at least one of a circulating_lymphoid class, a NHL_indolent class, a

NHL_aggressive class, a hodgkin_lymphoma class, a myeloid class, a plasma_cell class, a heme_1 class, and a heme_3 class. In some embodiments, the circulating_lymphoid class comprises one or more subclasses selected from the group consisting of hairy_cell_leukemia, low_grade_b_cell, lymphoplasmacytic, chronic lymphocytic leukemia (CLL), SLL, b_cell_lymphoblastic, and mantle_cell. In some embodiments, the NHL indolent class comprises one or more subclasses selected from the group consisting of MALT_NMZL and follicular_lymphoma. In some embodiments, the NHL_aggressive class comprises one or more subclasses selected from the group consisting of mature_t_cell_neoplasm, mediastinal_LBCL, high_grade_b_cell, and DLBCL. In some embodiments, the myeloid class comprises one or more subclasses selected from the group consisting of polycythemia vera (PV), MDS, CML, and AML. In some embodiments, the plasma_cell class comprises one or more subclasses selected from the group consisting of plasma_cell_neoplasm and plasma_cell_myeloma.

In some embodiments, the sequencing data comprise methylation sequencing data generated by methylation sequencing of the cfDNA fragments. In some embodiments, the methylation sequencing comprises WGBS. In some embodiments, the methylation sequencing comprises targeted sequencing. In some embodiments, the features derived from the methylation sequencing data are indicative of methylation patterns, clonal fraction, or rate of growth or turnover.

In some embodiments, the plurality of tissue of origin classes comprise one or more solid or liquid cancerous tissues of origin selected from the group consisting of: breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia. In some embodiments, the plurality of tissue of origin classes comprise a non-cancer class.

In other aspects, the present disclosure describes methods and systems for detecting and classifying cancer, wherein the method or system comprises receiving sequencing data for a biological sample comprising cfDNA fragments; analyzing the sequencing data using a multiclass classifier based on features derived from the sequencing data, wherein the multiclass classifier predicts a probability likelihood for each of a plurality of tissue of origin classes, the plurality of tissue of origin classes comprising one or more cancer tissue of origin classes and one or more hematological tissue of origin subtype classes; and determining, based on the probability likelihoods predicted by the multiclass classifier, the cancer classification, wherein the cancer classification comprises a presence or absence of cancer, a cancer tissue of origin, or a hematological tissue of origin.

In other embodiments, a method for predicting a presence or absence of cancer in a test sample comprises: accessing the test sample having a cancer score and a tissue signal for a first tissue label; selecting one of a plurality of strata based on the tissue signal for the first tissue label, the plurality of strata including a high signal stratum for the first tissue label and a low signal stratum of for the first tissue label; and predicting whether the test sample is associated with a presence or absence of cancer by comparing the cancer score against a binary threshold cutoff for the selected stratum.

In some embodiments, the test sample comprises a test feature vector determined according to methylation sequencing data of the test sample.

In some embodiments, the cancer score is determined by applying a binary cancer classifier to the test feature vector.

In some embodiments, the tissue signal is a tissue of origin (TOO) prediction determined by applying a multiclass cancer classifier to the test feature vector.

In some embodiments, the TOO prediction comprises a prediction value for each of a plurality of tissue labels, each prediction value indicating a likelihood that the test sample corresponds to a cancer type associated with the tissue label.

In some embodiments, selecting one of a plurality of strata based on the tissue signal for the first tissue label comprises: determining whether the tissue signal for the first tissue label is at or above a prediction value threshold; responsive to determining that the tissue signal for the first tissue label is at or above the prediction value threshold, selecting the high signal stratum; and responsive to determining that the tissue signal for the first tissue label is below the prediction value threshold, selecting the low signal stratum.

In some embodiments, the TOO prediction indicates one or more top predictions of one or more tissue labels of the plurality of tissue labels, wherein a top prediction of a tissue label indicates that the test sample is predicted to have a cancer type associated with the tissue label of the top prediction.

In some embodiments, selecting one of the plurality of strata comprises: determining whether the first tissue label is a top prediction; responsive to determining that the first tissue label is the top prediction, selecting the high signal stratum; and responsive to determining that the first tissue label is not the top prediction, selecting the low signal stratum.

In some embodiments, selecting one of a plurality of strata comprises: determining whether the first tissue label is a second top prediction; responsive to determining that the first tissue label is the second top prediction, selecting the high signal stratum; and responsive to determining that the first tissue label is not the second top prediction, selecting the low signal stratum.

In some embodiments, the plurality of strata includes a medium signal strata for a medium tissue signal.

In some embodiments, the test sample has a tissue signal for a second tissue class, wherein selecting one of a plurality of strata is further based on the tissue signal for the second tissue label.

In some embodiments, the binary threshold cutoff for each stratum is determined by: obtaining a holdout set of samples, each sample having a cancer score and a tissue signal for the first tissue label; stratifying the holdout set into the plurality of strata based on the tissue signals for the first tissue label of the holdout set of samples; for each stratum of the plurality of strata: sweeping through a domain of cancer scores at a plurality of candidate binary threshold cutoffs by calculating a true positive rate and a false positive rate for each candidate binary threshold cutoff based on the cancer scores of the samples in the stratum, and selecting a binary threshold cutoff from the plurality of candidate binary threshold cutoffs for the stratum based on a false positive budget for the stratum and the calculated false positive rates.

In other embodiments, a method is disclosed for detecting and classifying cancer, the method comprising: receiving sequencing data for a biological sample comprising cfDNA fragments; applying a multiclass classifier to features derived from the sequencing data, wherein the multiclass classifier predicts a probability likelihood for each of a plurality of hematological tissue of origin subtype classes; and determining, based on the probability likelihoods predicted by the multiclass classifier, a hematological tissue of origin associated with the biological sample. In some embodiments, a system comprising a hardware processor and a non-transitory computer-readable storage medium storing executable instructions that, when executed by the hardware processor, cause the processor to perform steps of the method.

In other embodiments, the multiclass classifier further predicts a probability

likelihood for a non-cancer class.

In other embodiments, the multiclass classifier is trained on training samples derived from samples with hematological conditions and non-cancer samples.

### BRIEF DESCRIPTION OF DRAWINGS

FIG. 1A is a flowchart describing a process of sequencing a fragment of cell-free (cf) DNA to obtain a methylation state vector, according to an embodiment.

FIG. 1B is an illustration of the process of FIG. 1A of sequencing a fragment of cell-free (cf) DNA to obtain a methylation state vector, according to an embodiment.

FIGS. 2A & 2B illustrate flowcharts describing a process of determining anomalously methylated fragments from a sample, according to an embodiment.

FIG. 3A is a flowchart describing a process of training a cancer classifier, according to an embodiment.

FIG. 3B illustrates an example generation of feature vectors used for training the cancer classifier, according to an embodiment.

FIG. 4A illustrates a flowchart of devices for sequencing nucleic acid samples according to one embodiment.

FIG. 4B is a block diagram of an analytics system, according to an embodiment.

FIG. 5 illustrates many graphs showing cancer prediction accuracy of a multiclass cancer classifier for various cancer types, according to an example implementation.

FIG. 6 illustrates many graphs showing cancer prediction accuracy of a multiclass cancer classifier for various cancer types after first using a binary cancer classifier, according to an example implementation.

FIG. 7 illustrates a confusion matrix demonstrating performance of a trained cancer classifier, according to an example implementation.

FIG. 8 illustrates a graph of cancer type likelihood for non-cancer samples above 95% specificity.

FIGS. 9A and 9B illustrate graphs of hematological subtypes separated according to methylation sequencing data.

FIG. 10A illustrates a flowchart describing a process of determining a binary threshold cutoff for binary cancer classification, in accordance with one or more embodiments.

FIG. 10B illustrates a flowchart describing a process of thresholding a TOO label for determining a binary threshold cutoff for binary cancer classification, in accordance with one or more embodiments.

FIG. 11 illustrates a confusion matrix demonstrating performance of a trained cancer tissue of origin classifier with additional hematological cancer subtypes.

FIGS. 12A and 12B illustrate graphs showing cancer prediction accuracy for cancer classifiers with and without adjusting a threshold cutoff for numerous cancer types over stages of cancer.

FIG. 13A illustrates a process for stratifying hematological signals into two strata, in accordance with one or more embodiments.

FIG. 13B illustrates a process for stratifying hematological signals into three strata, in accordance with one or more embodiments.

FIG. 13C illustrates a process for first stratifying hematological signals, and subsequently stratifying colorectal signals, in accordance with one or more embodiments.

FIG. 14 illustrates a process of determining binary threshold cutoffs for TOO stratification, in accordance with one or more embodiments.

FIG. 15 illustrates a flowchart describing a process of predicting cancer presence or cancer absence for a test sample using a binary threshold cutoff determined by TOO stratification, in accordance with one or more embodiments.

FIG. 16A illustrates a graph showing the classifier's sensitivity at 99.5% specificity level across the hematological subtypes.

FIG. 16B illustrates a graph showing the classifier's sensitivity at 95% specificity across stages for Hodgkin lymphomas and Non-Hodgkin lymphomas.

FIG. 17 illustrates a confusion matrix showing cancer prediction accuracy of the hematological-specific cancer classifier, in a first example implementation.

FIG. 18 illustrates a series of graphs plotting cancer score against distance from the centroid in the UMAP embedding for hematological-specific cancer classification, in the first example implementation.

FIG. 19 illustrates a graph plotting the anomaly scores of a plurality of training samples for hematological-specific cancer classification, in a second example implementation.

FIG. 20 illustrates a graph showing the hematological-specific cancer classifier's sensitivity at 99.5% specificity, in the second example implementation.

FIG. 21 illustrates a confusion matrix showing cancer prediction accuracy of the hematologic-specific cancer classifier, in the second example implementation.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

### I.A. Overview Of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of anomalously methylated cfDNA fragments. First off, determining a DNA fragment to be anomalously methylated only holds weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site causally influences methylation at a subsequent CpG site. To encapsulate this dependency is another challenge in itself.

Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation is characterized for a DNA fragment, if the DNA fragment comprises more than a threshold number of CpG sites with more than a threshold percentage of those CpG sites being methylated or unmethylated.

Those of skill in the art will appreciate that the principles described herein are equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein are the same, and consequently the inventive concepts described herein are applicable to those other forms of methylation.

### I.B. Definitions

The term “individual” refers to a human individual. The term “healthy individual” refers to an individual presumed to not have a cancer or disease. The term “subject” refers to an individual who is known to have, or potentially has, a cancer or disease.

The term “cell free nucleic acid” or “cfNA” refers to nucleic acid fragments that circulate in an individual's body (e.g., blood) and originate from one or more healthy cells and/or from one or more cancer cells. The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “sequence read” refers to a nucleotide sequence obtained from a nucleic acid molecule from a test sample from an individual. Sequence reads can be obtained through various methods known in the art.

The term “sequencing depth” or “depth” refers to a total number of sequence reads or read segments at a given genomic location or loci from a test sample from an individual.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

### II. Sample Processing

### II.A. Generating Methylation State Vectors for DNA Fragments

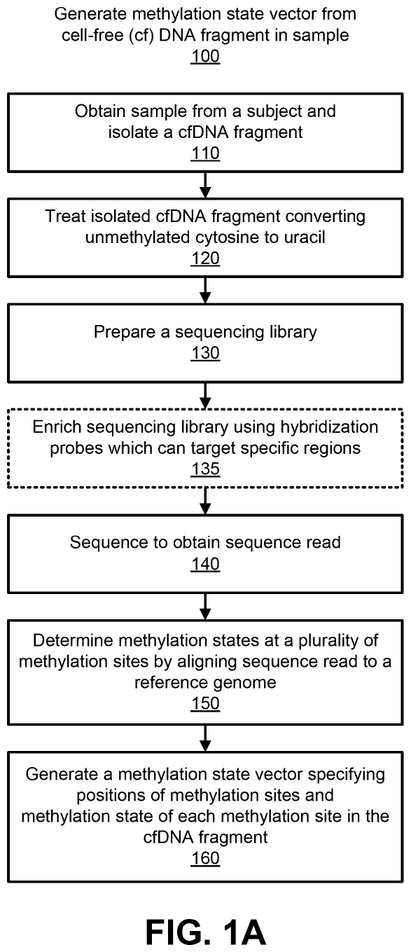

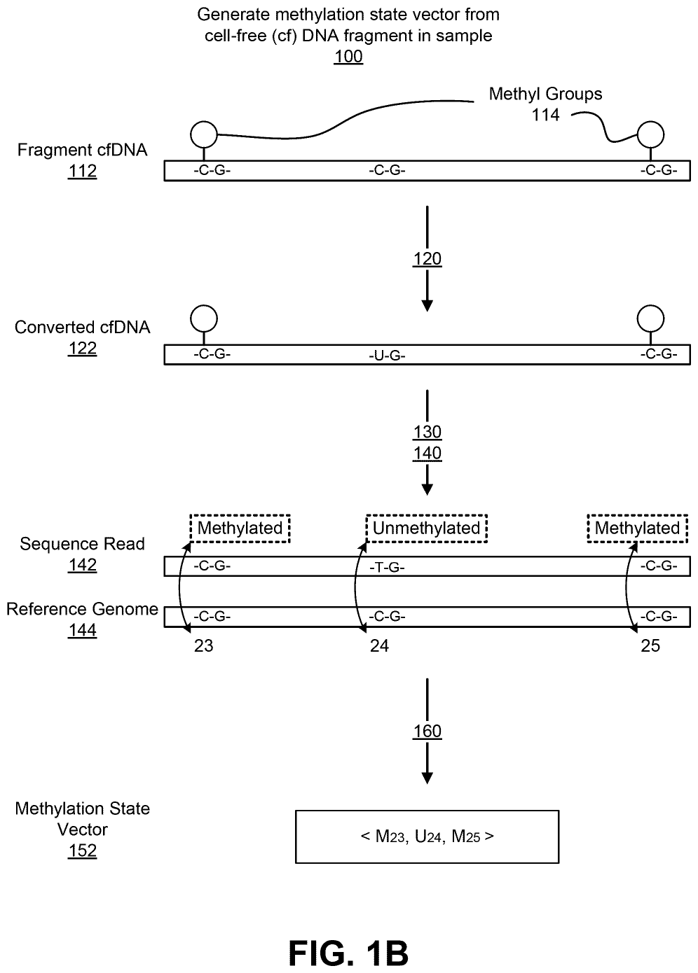

FIG. 1A is a flowchart describing a process 100 of sequencing a fragment of cell-free (cf) DNA to obtain a methylation state vector, according to an embodiment. In order to analyze DNA methylation, an analytics system first obtains 110 a sample from an individual comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From the sample, the analytics system isolates each cfDNA molecule. The cfDNA molecules are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library is prepared 130. Optionally, the sequencing library may be enriched 135 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. In one embodiment, the hybridization probes are designed to enrich for DNA molecules that have been treated (e.g., using bisulfite) for conversion of unmethylated cytosines to uracils. Once prepared, the sequencing library or a portion thereof can be sequenced to obtain a plurality of sequence reads. The sequence reads may be in a computer-readable, digital format for processing and interpretation by computer software.

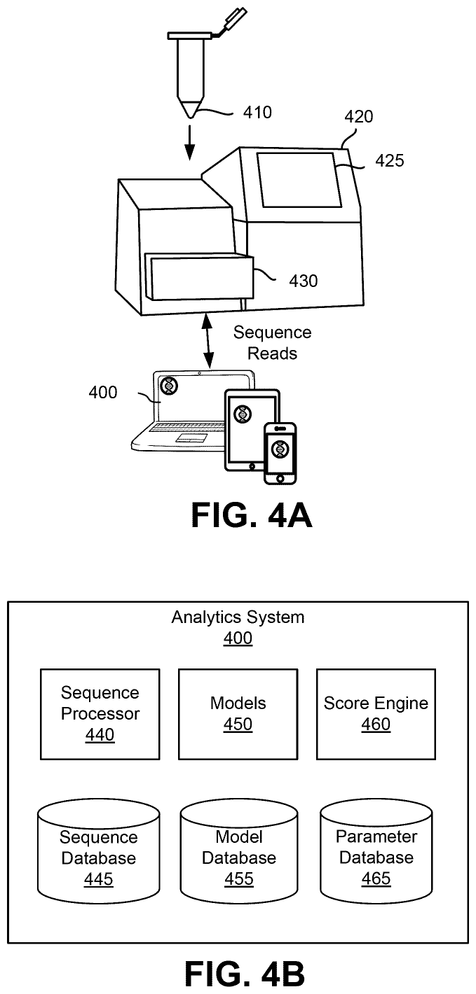

From the sequence reads, the analytics system determines 150 a location and methylation state for each CpG site based on alignment to a reference genome. The analytics system generates 160 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses; one such model will be described below in conjunction with FIG. 4.

FIG. 1B is an illustration of the process 100 of FIG. 1A of sequencing a cfDNA molecule to obtain a methylation state vector, according to an embodiment. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system thus generates information both on methylation status of all CpG sites on the cfDNA molecule 112 and the position in the human genome that the CpG sites map to. As shown, the CpG sites on sequence read 142 which were methylated are read as cytosines. In this example, the cytosines appear in the sequence read 142 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

The methylation-state vectors produced at step 160, and the sparse CpG-count matrices that aggregate those vectors for downstream analysis are entirely human-engineered data constructs. Each vector arises only after (i) chemical conversion of cytosines, (ii) laboratory library preparation, (iii) high-throughput sequencing, and (iv) computational alignment to an indexed reference genome. These cumulative transformations sever any direct connection to the naturally occurring cfDNA fragments in the bloodstream. The vector exists only in computer memory as an ordered list of symbols that encode genomic position and methylation state. Because neither the methylation-state vector nor the subsequent matrix can be found in nature or extracted without human intervention, the claimed subject matter concerns an artificial representation, not the underlying biological molecule or any natural correlation.

Moreover, the features vectors and matrices described herein are often manipulated in a manner for efficient processing and they oftentimes do not represent the sequenced data in a 1:1 manner and are therefore non-natural. For example, for training the classifier, the system augments empirical sequencing runs with in-silico constructs that recombine observed methylation patterns, rescale fragment counts, inject simulated read noise, etc. so that the aggregate training corpus matches predefined class and signal distributions. Each augmented sample therefore reflects a statistical synthesis performed in software rather than a physical cfDNA molecule. By feeding the classifiers with engineered examples, the system makes determinations without non-naturally occurring sequences or phenomenon.

### II.B. Identifying Anomalous Fragments

The analytics system determines anomalous fragments for a sample using the sample's methylation state vectors. For each fragment in a sample, the analytics system determines whether the fragment is an anomalous fragment using the methylation state vector corresponding to the fragment. In one embodiment, the analytics system calculates a p-value score for each methylation state vector describing a probability of observing that methylation state vector or other methylation state vectors even less probable in the healthy control group. The process for calculating a p-value score will be further discussed below in Section II.B.i. P-Value Filtering. The analytics system may determine fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### II.B.I. P-Value Filtering

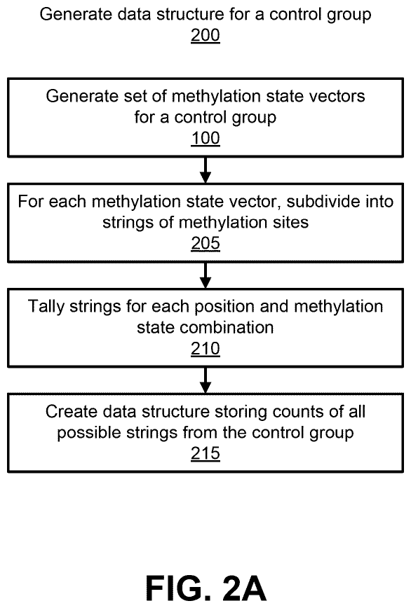

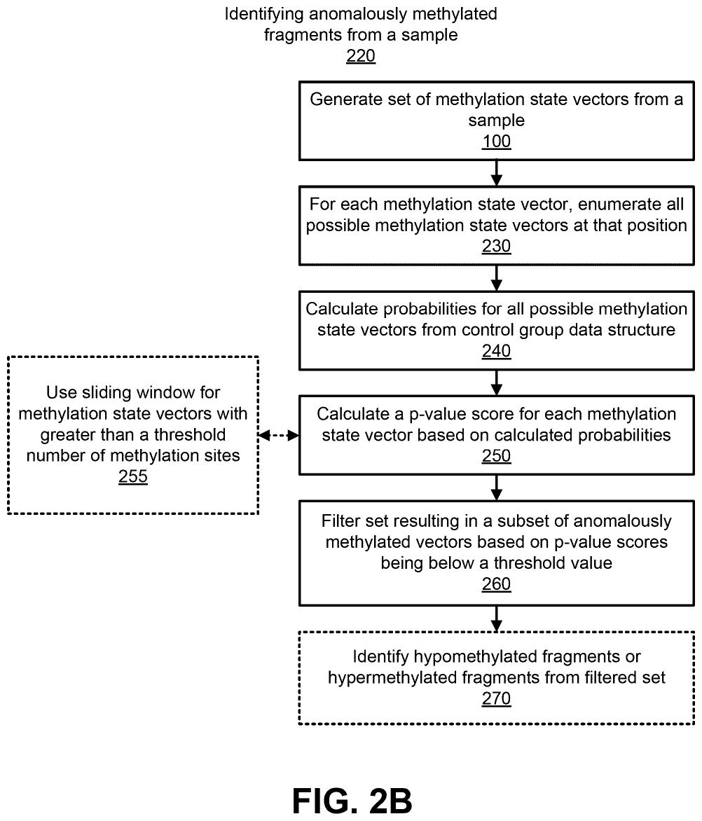

In one embodiment, the analytics system calculates a p-value score for each methylation state vector compared to methylation state vectors from fragments in a healthy control group. The p-value score describes a probability of observing the methylation status matching that methylation state vector or other methylation state vectors even less probable in the healthy control group. In order to determine a DNA fragment to be anomalously methylated, the analytics system uses a healthy control group with a majority of fragments that are normally methylated. When conducting this probabilistic analysis for determining anomalous fragments, the determination holds weight in comparison with the group of control subjects that make up the healthy control group. To ensure robustness in the healthy control group, the analytics system may select some threshold number of healthy individuals to source samples including DNA fragments. FIG. 2A below describes the method of generating a data structure for a healthy control group with which the analytics system may calculate p-value scores. FIG. 2B describes the method of calculating a p-value score with the generated data structure.

FIG. 2A is a flowchart describing a process 200 of generating a data structure for a healthy control group, according to an embodiment. To create a healthy control group data structure, the analytics system receives a plurality of DNA fragments (e.g., cfDNA) from a plurality of healthy individuals. A methylation state vector is identified for each fragment, for example via the process 100.

With each fragment's methylation state vector, the analytics system subdivides 205 the methylation state vector into strings of CpG sites. In one embodiment, the analytics system subdivides 205 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 210 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 210 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 215 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it requires a significant amount of data that may not be available, and thus would be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is anomalous or not.

FIG. 2B is a flowchart describing a process 220 for identifying anomalously methylated fragments from an individual, according to an embodiment. In process 220, the analytics system generates 100 methylation state vectors from cfDNA fragments of the subject. The analytics system handles each methylation state vector as follows.

For a given methylation state vector, the analytics system enumerates 230 all

possibilities of methylation state vectors having the same starting CpG site and same length (i.e., set of CpG sites) in the methylation state vector. As each methylation state is generally either methylated or unmethylated there are effectively two possible states at each CpG site, and thus the count of distinct possibilities of methylation state vectors depends on a power of 2, such that a methylation state vector of length n would be associated with 2ⁿ possibilities of methylation state vectors. With methylation state vectors inclusive of indeterminate states for one or more CpG sites, the analytics system may enumerate 230 possibilities of methylation state vectors considering only CpG sites that have observed states.

The analytics system calculates 240 the probability of observing each possibility of methylation state vector for the identified starting CpG site and methylation state vector length by accessing the healthy control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system calculates 250 a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The analytics system sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy individual, and which causes the fragment to be labeled anomalously methylated, relative to the healthy control group. A high p-value score generally relates to a methylation state vector is expected to be present, in a relative sense, in a healthy individual. If the healthy control group is a non-cancerous group, for example, a low p-value indicates that the fragment is anomalous methylated relative to the non-cancer group, and therefore possibly indicative of the presence of cancer in the test subject.

As above, the analytics system calculates p-value scores for each of a plurality of methylation state vectors, each representing a cfDNA fragment in the test sample. To identify which of the fragments are anomalously methylated, the analytics system may filter 260 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

According to example results from the process 400, the analytics system yields a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below in Section III.

In one embodiment, the analytics system uses 255 a sliding window to determine possibilities of methylation state vectors and calculate p-values. Rather than enumerating possibilities and calculating p-values for entire methylation state vectors, the analytics system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The analytics system then “slides” the window to the second CpG site in the vector, and calculates another p-value score for the second window. Thus, for a window size l and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the analytics system aggregates the p-value scores for the methylation state vectors to generate an overall p-value score.

Using the sliding window helps to reduce the number of enumerated possibilities of methylation state vectors and their corresponding probability calculations that would otherwise need to be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system identifies all possibilities that have consensus with the all methylation states of the methylation state vector excluding the indeterminate states. The analytics system may assign the probability to the methylation state vector as a sum of the probabilities of the identified possibilities. As an example, the analytics system calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states uses calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

### II.B.II. Hypermethylated Fragments and Hypomethylated Fragments

In another embodiment, the analytics system determines anomalous fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### II.C. Example Analytics System

FIG. 4A is a flowchart of devices for sequencing nucleic acid samples according to one embodiment. This illustrative flowchart includes devices such as a sequencer 420 and an analytics system 400. The sequencer 420 and the analytics system 400 may work in tandem to perform one or more steps in the processes 100 of FIG. 1A, 200 of FIG. 2A, 220 of FIG. 2B, and other process described herein.

In various embodiments, the sequencer 420 receives an enriched nucleic acid sample

410. As shown in FIG. 4A, the sequencer 420 can include a graphical user interface 425 that enables user interactions with particular tasks (e.g., initiate sequencing or terminate sequencing) as well as one more loading stations 430 for loading a sequencing cartridge including the enriched fragment samples and/or for loading necessary buffers for performing the sequencing assays. Therefore, once a user of the sequencer 420 has provided the necessary reagents and sequencing cartridge to the loading station 430 of the sequencer 420, the user can initiate sequencing by interacting with the graphical user interface 425 of the sequencer 420. Once initiated, the sequencer 420 performs the sequencing and outputs the sequence reads of the enriched fragments from the nucleic acid sample 410.

In some embodiments, the sequencer 420 is communicatively coupled with the analytics system 400. The analytics system 400 includes some number of computing devices used for processing the sequence reads for various applications such as assessing methylation status at one or more CpG sites, variant calling or quality control. The sequencer 420 may provide the sequence reads in a BAM file format to the analytics system 400. The analytics system 400 can be communicatively coupled to the sequencer 420 through a wireless, wired, or a combination of wireless and wired communication technologies. Generally, the analytics system 400 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information, e.g., via step 140 of the process 100 in FIG. 1A. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 400 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary) format may be generated and output for further analysis.

Referring now to FIG. 4B, FIG. 4B is a block diagram of an analytics system 400 for processing DNA samples according to one embodiment. The analytics system implements one or more computing devices for use in analyzing DNA samples. The analytics system 400 includes a sequence processor 440, sequence database 445, model database 455, models 450, parameter database 465, and score engine 460. In some embodiments, the analytics system 400 performs some or all of the processes 100 of FIG. 1A and 200 of FIG. 2.

The sequence processor 440 generates methylation state vectors for fragments from a sample. At each CpG site on a fragment, the sequence processor 440 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 1A. The sequence processor 440 may store methylation state vectors for fragments in the sequence database 445. Data in the sequence database 445 may be organized such that the methylation state vectors from a sample are associated to one another.

Further, multiple different models 450 may be stored in the model database 455 or retrieved for use with test samples. In one example, a model is a trained cancer classifier for determining a cancer prediction for a test sample using a feature vector derived from anomalous fragments. The training and use of the cancer classifier will be further discussed in conjunction with Section III. Cancer Classifier for Determining Cancer. The analytics system 400 may train the one or more models 450 and store various trained parameters in the parameter database 465. The analytics system 400 stores the models 450 along with functions in the model database 455.

During inference, the score engine 460 uses the one or more models 450 to return outputs. The score engine 460 accesses the models 450 in the model database 455 along with trained parameters from the parameter database 465. According to each model, the score engine receives an appropriate input for the model and calculates an output based on the received input, the parameters, and a function of each model relating the input and the output. In some use cases, the score engine 460 further calculates metrics correlating to a confidence in the calculated outputs from the model. In other use cases, the score engine 460 calculates other intermediary values for use in the model.

### III. Cancer Classifier for Determining Cancer

### III.A. Overview

Early detection of cancer is important because it improves patient outcomes, enhances treatment effectiveness, and can extend life expectancy of a patient. Timely and accurate cancer diagnoses offer considerable benefits not only to patients, but also to healthcare providers and family members. A patient's doctors and nurses can provide more targeted and effective care with early, accurate diagnoses, while caregivers experience reduced emotional and financial burdens when cancer is identified and managed earlier. Overall, a cancer diagnosis and its corresponding treatment process signifies a profound hardship in both a patient and adjacent persons' lives, and a corresponding increase in the capability and accuracy of the diagnoses process represents an ability to alleviate potential heartbreak.

Historically, cancer detection has relied upon imaging technologies such as physical analysis, CT scans, or X-rays, which identify tumors based on visible, physical characteristics within tissue structures. For example, mammograms detect abnormalities indicative of breast cancer by analyzing tissue densities and structural irregularities. While effective in some cases, these traditional diagnostic methods often require tumors to reach a detectable physical size before identification, limiting their ability to catch cancer in its earliest, most treatable stages.

Recent advances have significantly shifted cancer diagnostics toward methods based on the molecular analysis of genetic fragments, such as, for example, circulating cell-free DNA (cfDNA) found in a patient's bloodstream. Unlike traditional imaging methods, liquid-based analyses enable detection at the genomic level, identifying subtle molecular indicators of cancer long before physical tumors are visible through traditional imaging techniques or physical diagnoses. Consequently, these newer methodologies offer substantial advantages in terms of sensitivity, timeliness, specificity, and patient comfort-requiring only minimally invasive sampling rather than more invasive procedures.

In turn, the advanced cfDNA-based cancer classification technology disclosed in the present specification represents a technical improvement in cancer diagnostics. Specifically, the pipeline first applies a multi-class classifier to a sample-specific cfDNA data structure representing genomic features to generate a cancer score, a tissue-of-origin prediction, and a tissue-signal confidence value. The pipeline then uses the tissue-signal to place the sample in a high-signal or low-signal stratum associated with the predicted tissue, where a stratum-specific binary detector evaluates cancer presence. For each stratum, a threshold optimization strategy selects a binary cut-off that satisfies a predefined false-positive budget, thereby delivering calibrated decisions that reduce false positives for low-incidence tissues while maintaining early-stage sensitivity.

To that end, the cancer classifier is trained to receive a feature vector for a test sample and determine whether the test sample is from a test subject that has cancer or, more specifically, a particular cancer type. The cancer classifier comprises a plurality of classification parameters and a function representing a relation between the feature vector as input and the cancer prediction as output determined by the function operating on the input feature vector with the classification parameters. In one embodiment, the feature vectors input into the cancer classifier are based on set of anomalous fragments determined from the test sample. The anomalous fragments may be determined via the process 220 in FIG. 2B, or more specifically hypermethylated and hypomethylated fragments as determined via the step 270 of the process 220, or anomalous fragments determined according to some other process. Prior to deployment of the cancer classifier, the analytics system trains the cancer classifier with the process 300.

### III.B. Training of Cancer Classifier

As noted above, advances in modern cancer diagnostics increasingly rely on genomic analysis techniques. Many of those advances rely on high-throughput sequencing systems, some of which are descried hereinbelow. These systems generate comprehensive genomic datasets that allow identification and characterization of molecular markers of disease (e.g., cancer). Because they are high throughput, the sequencing systems facilitate parallel analysis of millions to billions of DNA fragments, significantly increasing both the scale and resolution of genomic investigations. These technological capabilities have enabled precise detection of various genomic attributes (e.g., genetic aberrations, differential methylation patterns, etc.) that can signify cancerous conditions. The detected genomic attributes enable early detection, classification, and monitoring of cancers from biological samples (e.g., samples and sequenced cfDNA).

Despite these capabilities, the data produced using high-throughput sequencing methods introduce numerous technical challenges. Genomic datasets obtained from sequencing can be extremely large, commonly comprising tens of millions to billions of nucleic acid fragments, each containing multiple genomic loci (e.g., terabytes of data). In addition to size and noise, sequencing datasets exhibit pronounced class imbalance: non-cancer samples outnumber cancer samples, and within the cancer cohort early-stage cases and certain tissue-of-origin subtypes occur infrequently. Handling, storing, and processing these vast datasets requires substantial computational resources and sophisticated analytical systems. Furthermore, sequencing procedures are inherently susceptible to errors, such as incorrect nucleotide calls (mistranscriptions), incomplete sequencing reactions, or inaccurate alignment to a reference genome. These errors can be compounded when there are significant imbalances in the data and can result in noisy or contaminated datasets, complicating the identification of reliable molecular signals necessary for accurate disease classification. Accordingly, the machine learning methods above implement a two-stage classification system that addresses some of the technical downfalls of sequencing data.

Additionally, training a model that addresses the complex technical problems associated with dataset quality, scale, and sufficiency is not a mental process practically performable by the human mind. The processing of genomic data from high-throughput sequencing systems requires rapid execution of thousands to millions of probabilistic calculations, iterative optimization processes on massive datasets, and high-dimensional data transformations. Human cognitive limitations inherently preclude real-time or even near-real-time interpretation and processing of such vast, complex genomic datasets. For instance, manually attempting the computations needed to identify anomalously methylated cfDNA fragments or training predictive classifiers at clinically relevant limits of detection would require impractically extensive periods of time, rendering any resulting patient diagnosis meaningless due to delays in clinical intervention.

Moreover, the disclosed techniques involving the training of predictive classifiers from genomic data do not merely constitute mathematical concepts. Neural networks and other machine learning models employed in training cancer classifiers rely on complex, non-linear transformations of genomic features into predictive outputs through multiple iterative computational layers. These transformations involve high-dimensional numeric computations, gradient optimizations, and backpropagation through potentially millions of connections across network nodes. Such procedures exceed basic mathematical relationships or formulas and represent sophisticated computational modeling of biological processes that cannot be reduced to mere abstract mathematical concepts or formulas alone.

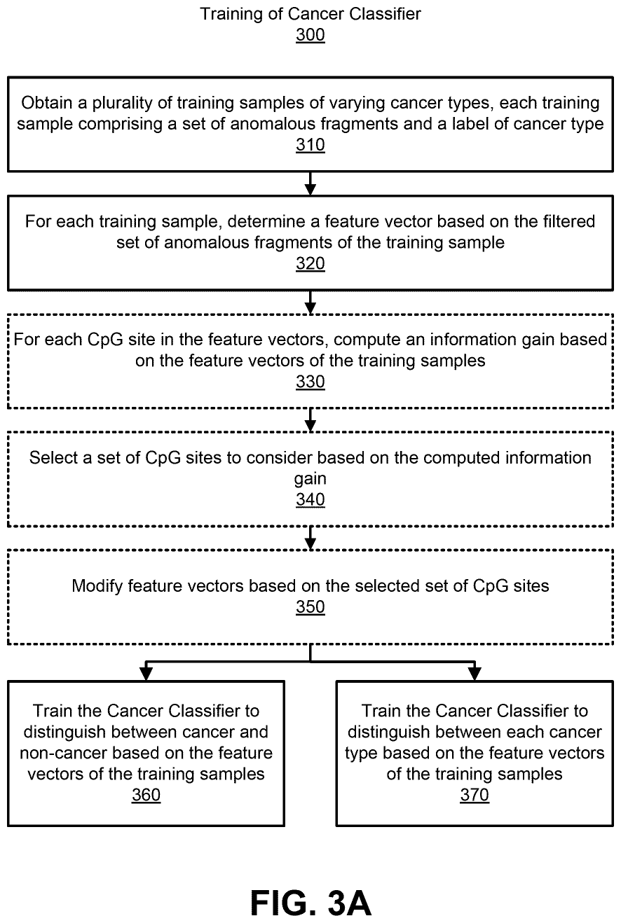

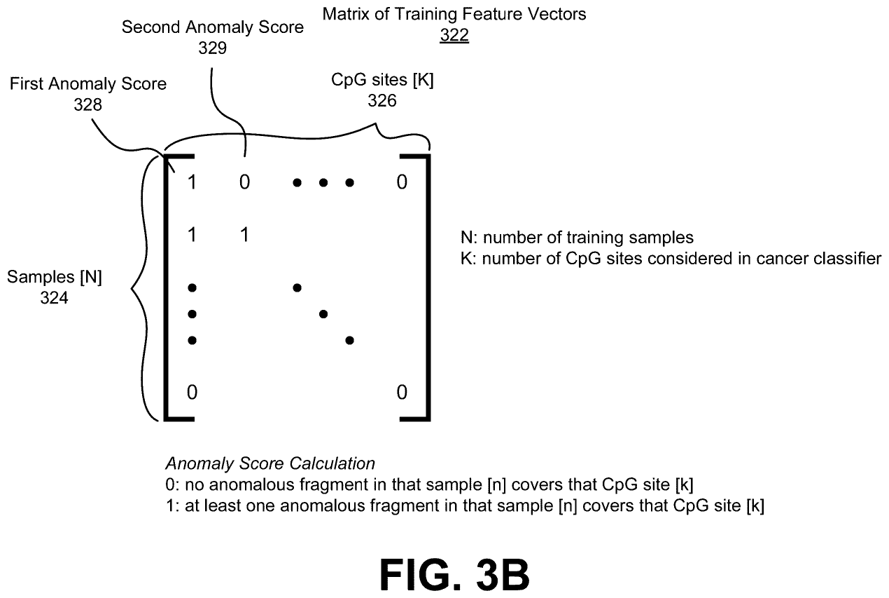

FIG. 3A is a flowchart describing a process 300 of training a cancer classifier, according to an embodiment. The analytics system obtains 310 a plurality of training samples each having a set of anomalous fragments and a label of a cancer type. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 320, for each training sample, a feature vector based on the set of anomalous fragments of the training sample. The analytics system calculates an anomaly score for each CpG site in an initial set of CpG sites. The initial set of CpG sites may be all CpG sites in the human genome or some portion thereof—which may be on the order of 104, 105, 106, 107, 108, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5.

Once all anomaly scores are determined for a training sample, the analytics system determines the feature vector as a vector of elements including, for each element, one of the anomaly scores associated with one of the CpG sites in an initial set. The analytics system normalizes the anomaly scores of the feature vector based on a coverage of the sample. Here, coverage refers to a median or average sequencing depth over all CpG sites covered by the initial set of CpG sites used in the classifier, or based on the set of anomalous fragments for a given training sample.

As an example, reference is now made to FIG. 3B illustrating a matrix of training feature vectors 322. In this example, the analytics system has identified CpG sites [K] 326 for consideration in generating feature vectors for the cancer classifier. The analytics system selects training samples [N] 324. The analytics system determines a first anomaly score 328 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 328 for the first CpG site as 1, as illustrated in FIG. 3B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 329 for the second CpG site [k2] to be 0, as illustrated in FIG. 3B. Once the analytics system determines all the anomaly scores for the initial set of CpG sites, the analytics system determines the feature vector for the first training sample [n1] including the anomaly scores with the feature vector including the first anomaly score 328 of 1 for the first CpG site [k1] and the second anomaly score 329 of 0 for the second CpG site [k2] and subsequent anomaly scores, thus forming a feature vector [1, 0, . . . ].

The analytics system may further limit the CpG sites considered for use in the cancer classifier. The analytics system computes 330, for each CpG site in the initial set of CpG sites, an information gain based on the feature vectors of the training samples. From step 320, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 330 an information gain for each cancer type and for each CpG site in the initial set to determine whether to include that CpG site in the classifier. The information gain is computed for training samples with a given cancer type compared to all other samples. For example, two random variables ‘anomalous fragment’ (‘AF’) and ‘cancer type’ (‘CT’) are used. In one embodiment, AF is a binary variable indicating whether there is an anomalous fragment overlapping a given CpG site in a given samples as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. In practice, for a first cancer type, the analytics system computes pairwise mutual information gain against each other cancer type and sums the mutual information gain across all the other cancer types.

For a given cancer type, the analytics system uses this information to rank CpG sites based on how cancer specific they are. This procedure is repeated for all cancer types under consideration. If a particular region is commonly anomalously methylated in training samples of a given cancer but not in training samples of other cancer types or in healthy training samples, then CpG sites overlapped by those anomalous fragments will tend to have high information gains for the given cancer type. The ranked CpG sites for each cancer type are greedily added (selected) 340 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 350 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 320 or to the selected set of CpG sites from step 350. In one embodiment, the analytics system trains 360 a binary cancer classifier to distinguish between cancer and non-cancer based on the feature vectors of the training samples. In this manner, the analytics system uses training samples that include both non-cancer samples from healthy individuals and cancer samples from subjects. Each training sample has one of the two labels “cancer” or “non-cancer.” In this embodiment, the classifier outputs a cancer prediction indicating the likelihood of the presence or absence of cancer.

In another embodiment, the analytics system trains 450 a multiclass cancer classifier to distinguish between many cancer types (also referred to as tissue of origin (TOO) labels). Cancer types include one or more cancers and may include a non-cancer type (may also include any additional other diseases or genetic disorders, etc.). To do so, the analytics system uses the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier is sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

### III.C. Tuning of Cancer Classifier

As noted above, early cancer-detection efforts have improved greatly over the recent years. Indeed they have progressed from size-limited imaging modalities toward molecular analyses that interrogate circulating cell-free DNA (cfDNA). Molecular assays provide sequence-level signals that appear before a tumor becomes radiographically apparent, yet the resulting data sets are high-dimensional and highly imbalanced across cancer sub-types. Generic machine-learning (ML) pipelines struggle with this imbalance, producing elevated false-positive rates for low-incidence tissues and diminished sensitivity for early-stage disease.

The present system addresses these limitations through a hierarchical ML architecture that couples an initial multi-class classifier with a stratification step. The classifier assigns each sample (i) a cancer score, (ii) a tissue-of-origin (TOO) prediction, and (iii) a tissue-signal confidence value. The hierarchical arrangement uses the confidence value to select one of multiple strata (e.g., a high-signal or low-signal stratum) for the predicted TOO. Each stratum possesses its own binary cancer-detection model and an independently optimized threshold chosen to satisfy a predefined false-positive budget for that stratum. By isolating subsequent inference to a stratum matched to both tissue type and confidence level, the pipeline equalizes class distributions seen by the binary detectors and enables threshold settings that are more aggressive for high-signal strata and more conservative for low-signal strata.

As shown below, empirical evaluation demonstrates that the stratified approach improves model sensitivity and lowers false-positive rates relative to a single-stage baseline operating on the same cfDNA features. The improvement derives from a measurable ML advance (e.g., adaptive routing of TOO to stratum-specific models) rather than from the mere application of standard classifiers to genomic data. Because these machine learning enhancements manifest in reduced diagnostic error at earlier disease stages, the architecture constitutes a technical improvement in both machine-learning processing and cancer diagnostics, achieved without increasing sample collection burden or assay complexity.

To continue, during use of the cancer classifier, the analytics system may perform operations to tune the predictive capabilities of the cancer classifier. Generally, a sample distribution may include one or more non-cancer samples with high tissue signal. Some of these high tissue signal non-cancer samples may even be pre-stage cancer, early stage cancer, or undiagnosed cancer. As such, non-cancer samples with high-tissue signal may muddle the predictive capabilities of the cancer classifier.

As used herein, “high tissue signal” refers to a sample with a tissue signal, e.g., generally for any type of tissue or for a particular cancer type—also referred to as a TOO label, that exceeds some threshold. The tissue signal may be determined by a multiclass cancer classifier or other approaches, in comparison to a healthy distribution. Non-cancer samples with high tissue signal are outliers in the non-cancer distribution. Some of these non-cancer samples may be pre-stage cancer, early stage cancer, or undiagnosed cancer. The analytics system can identify non-cancer samples with high tissue signal in at least one TOO label. In one approach of determining high tissue signal, a prediction value for a TOO label output by the multiclass cancer classifier is compared against a tissue signal threshold. Samples with a prediction value above the tissue signal threshold are deemed to have high tissue signal for that TOO label; whereas, samples with a prediction value below the tissue signal threshold are deemed to not have high tissue signal for that TOO label (or low tissue signal). In another approach, one or more top predictions in a TOO prediction are considered. For example, a TOO prediction for a sample has a first prediction of the colorectal TOO label, a second prediction of the breast TOO label, and a third prediction of head/neck TOO label. If the top prediction is considered, then the sample is deemed to have high tissue signal for the TOO label in the first prediction, that being the colorectal TOO label in the example. If the top two predictions are considered, then there is high tissue signal in both the colorectal TOO label and the breast TOO label. Other approaches of determining tissue signal may include other models trained to determine tissue signal for one or more TOO labels. Such models may include classifiers trained to determine tissue signal for a subset of TOO labels. For example, a hematological-specific classifier may be trained and used to determine tissue signal for one or more hematological subtypes. Two such example implementations are described under Section V. Example Results of Cancer Classifier. Other models include deconvolution models that can deconvolve tissue signal from methylation sequencing data (and/or other types of sequencing data).

In one embodiment of tuning the classifier, excluding high tissue signal non-cancer samples improves the classifier's sensitivity in cancer prediction. With a pruned sample distribution, a binary threshold cutoff may be determined according to a minimum specificity, wherein the binary threshold cutoff is used to predict presence or absence of cancer in a test sample. This method is further elaborated under Section III.C.i. Removal of High Signal Non-Cancer Samples.

In another embodiment of tuning the cancer classifier, the sample distribution may be stratified according to TOO signal. The analytics system determines a binary threshold cutoff for each resulting stratum with the samples stratified into the stratum. With a test sample, the analytics system places the test sample into a stratum according to the TOO signal and predicts the presence or absence of cancer in the test sample with the stratum's binary threshold cutoff. This method is further elaborated under Section III.C.ii. Stratification of Sample Distribution According to TOO Signal.

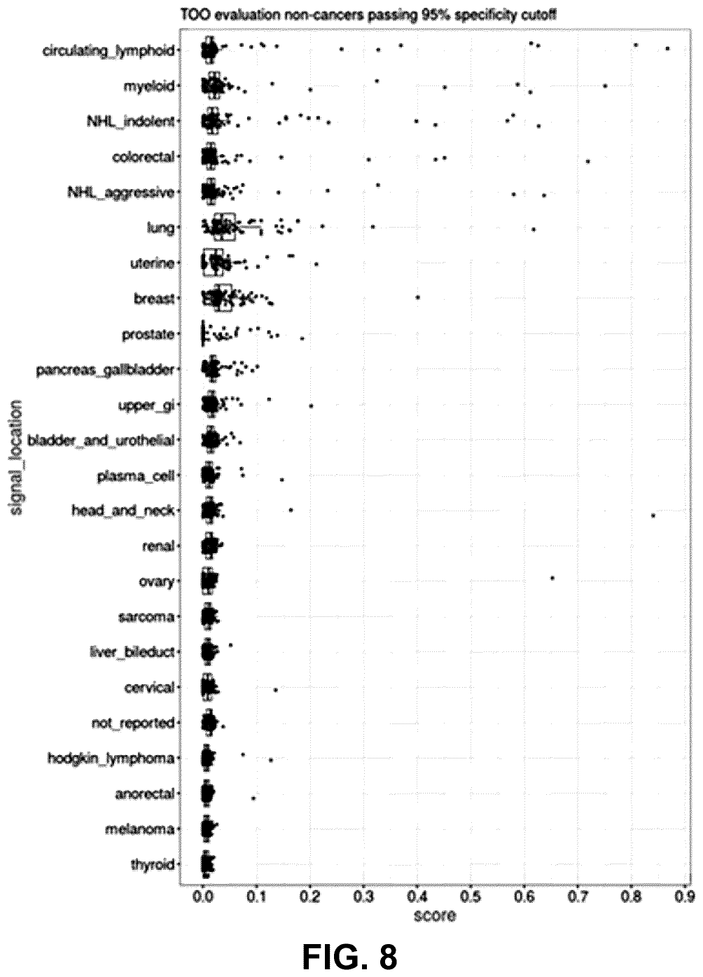

Referring to FIG. 8, FIG. 8 illustrates a graph of cancer type likelihood for non-cancer samples above 95% specificity. A cancer score was calculated for each non-cancer sample from a plurality of non-cancer samples, i.e., samples from healthy individuals not currently diagnosed with cancer. The cancer score can be determined by the binary classifier as a likelihood that a sample has cancer given the sample's methylation sequencing data. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data. One example of a classifier is a mixture model classifier. A distribution of the non-cancer samples can be generated according to the cancer scores of the non-cancer samples. A binary threshold cutoff can be set to ensure some level of binary classification specificity, e.g., a true negative rate. Typically, a high specificity cutoff is used in classifying cancer, e.g., 99.4% specificity or higher. However, many non-cancer samples, used in training the cancer classifier and just below the specificity cutoff, can have high tissue signal thereby positively biasing the binary threshold cutoff.

In some embodiments, certain cancer types are further separated into cancer subtypes. For example, the hematological cancer type can further be separated into a combination of, for instance, circulating lymphoid subtype, non-Hodgkin's-Lymphoma (NHL) indolent subtype, NHL aggressive subtype, Hodgkin's-Lymphoma (HL) subtype, myeloid subtype, and plasma cell subtype, all of which also belong to a lymphoid neoplasm class.

To demonstrate, non-cancer samples above the 95% specificity were selected and then input into a multiclass cancer classifier to determine a probability for each cancer type—or tissue of origin (TOO). The cancer types or TOO labels used in this embodiment of the multiclass cancer classifier include circulating lymphoid, myeloid, NHL indolent, colorectal, NHL aggressive, lung, uterine, breast, prostate, pancreas and gallbladder, upper gastrointestinal, bladder and urothelial, plasma cell, head and neck, renal, ovary, sarcoma, liver and bile duct, cervical, other tissues, HL, anorectal, melanoma, thyroid. The graph in FIG. 8 shows many non-cancer samples having high tissue signal from at least one tissue type. Each dot in a row for a tissue type corresponds to a tissue of origin likelihood for a non-cancer sample above the 95% specificity threshold. Notably, many tissue types have multiple non-cancer sample outliers having significant tissue contribution, not typical for non-cancer samples. This can arise when such non-cancer samples have cfDNA signals being driven by cancer-like methylation, clonal fraction, and/or rate of growth/turnover. Nonetheless, these non-cancer samples with significant tissue contribution shift the binary classification threshold cutoff up thereby decreasing sensitivity of the cancer classification, especially with samples with significant tissue signal just below the previously set binary classification threshold cutoff. In practice, such signals (e.g., corresponding to circulating_lymphoid, myeloid, and NHL_indolent) can be a major attractor of false positive determinations. Of note, circulating lymphoid, myeloid, NHL indolent, colorectal, NHL aggressive, lung, uterine, breast, prostate, pancreas and gallbladder, upper gastrointestinal, plasma cell, head and neck, cervical, HL had at least one non-cancer sample with a probability of tissue origin above 0.1. Particularly, circulating lymphoid, myeloid, NHL indolent, and NHL aggressive (all hematological subtypes) had two or more non-cancer samples with a probability of tissue origin above 0.5.

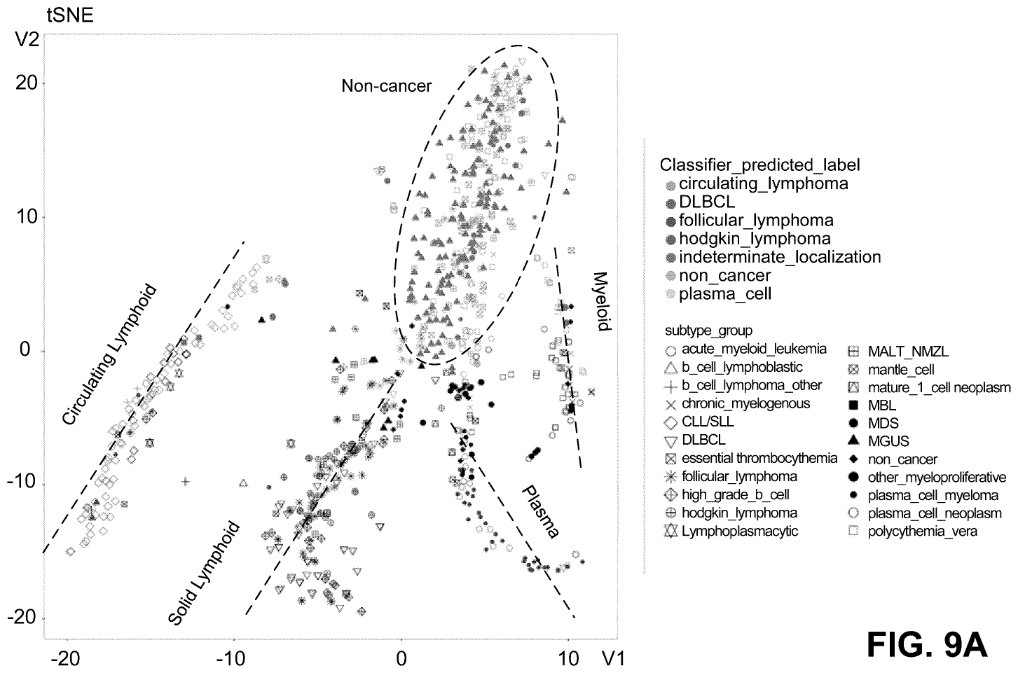

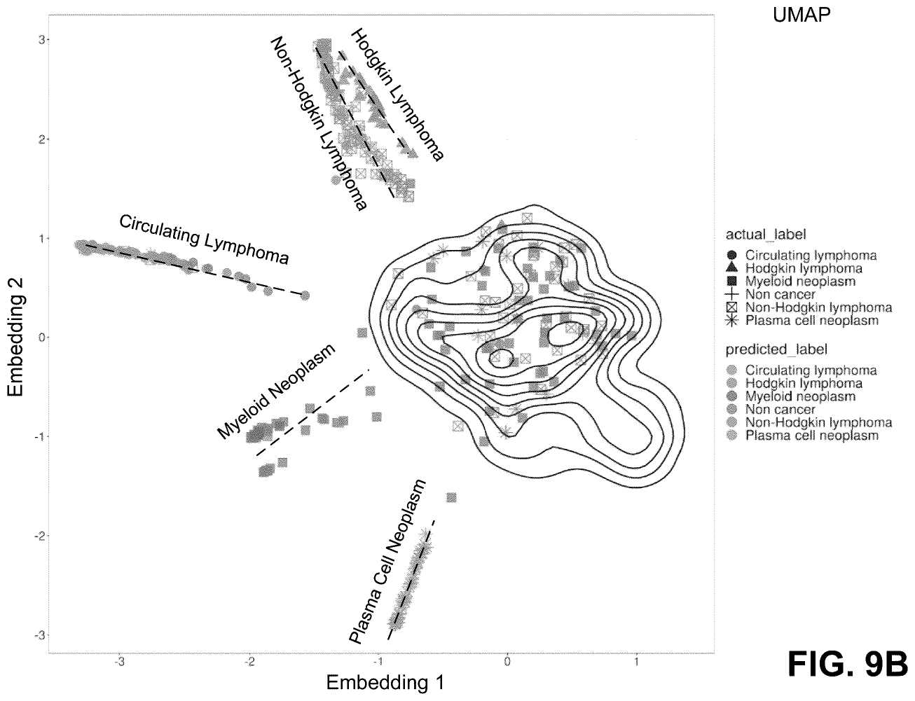

Referring to FIGS. 9A and 9B, FIGS. 9A and 9B illustrate graphs of hematological subtypes separated according to methylation sequencing data. The graphs of FIGS. 9A and 9B demonstrate an ability to model hematological subtypes. This can prove beneficial in providing more granularity to the multiclass cancer classification (e.g., classifying additionally with the hematological subtype labels) or as a manner of tuning the cancer classification through pruning non-cancer samples with high hematological subtype signal prior to training the cancer classifier. As described above, methylation signal can cover a plurality of CpG sites, thereby creating a high-dimensional vector space. The hematological subtypes shown include circulating lymphoid, solid lymphoid, plasma cell, and myeloid. The solid lymphoid subtype can be further divided into HL, NHL indolent, and NHL aggressive.

In FIG. 9A, the analytics system performs a t-distributed stochastic neighbor embedding. The t-distributed stochastic neighbor embedding identifies reduced dimensionality of the vector space (encompassing the methylation sequencing data) into a smaller number of embeddings. The embeddings are in order of variance in methylation signal amongst the samples. The first principal embedding, shown as “V1” on the horizontal axis on the graph, has the highest variance. The third principal embedding, shown as “V2” on the vertical axis on the graph, has the third highest variance. Annotated on the graph are clusters of the samples for each hematological subtype and non-cancer.

In FIG. 9B, the analytics system performs a UMAP embedding. The UMAP embedding also reduces dimensionality of the vector space into a smaller number of embeddings. The embeddings are in order of variance in methylation signal amongst the samples. The first principal embedding, shown as “embedding 1” on the horizontal axis on the graph, has the highest variance. The second principal embedding, shown as “embedding 2” on the vertical axis on the graph, has the second highest variance. Non-cancer samples are shown using a contour density.

The graphs show potential for classifying according to the hematological subtypes-either for addition of the hematological subtypes in the multiclass cancer classification or for modeling each of the hematological subtypes for tuning of the cancer classifiers.

### III.C.I. Removal of High Signal Non-Cancer Samples

In one or more embodiments, the analytics system tunes the trained cancer classifier by pruning the non-cancer samples used in training the cancer classifier. In particular, the analytics system may seek to remove non-cancer samples with high tissue signal that dilute the cancer classifier's sensitivity in cancer prediction.

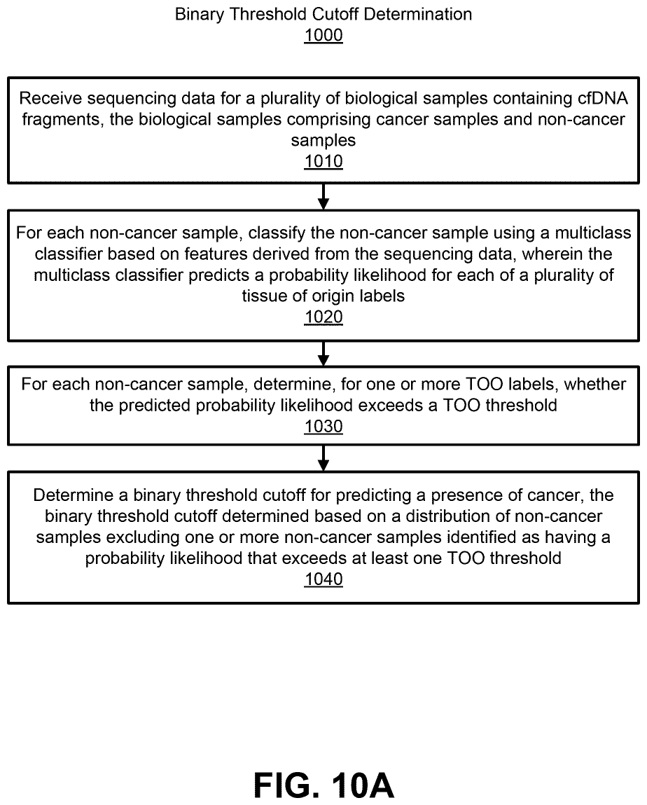

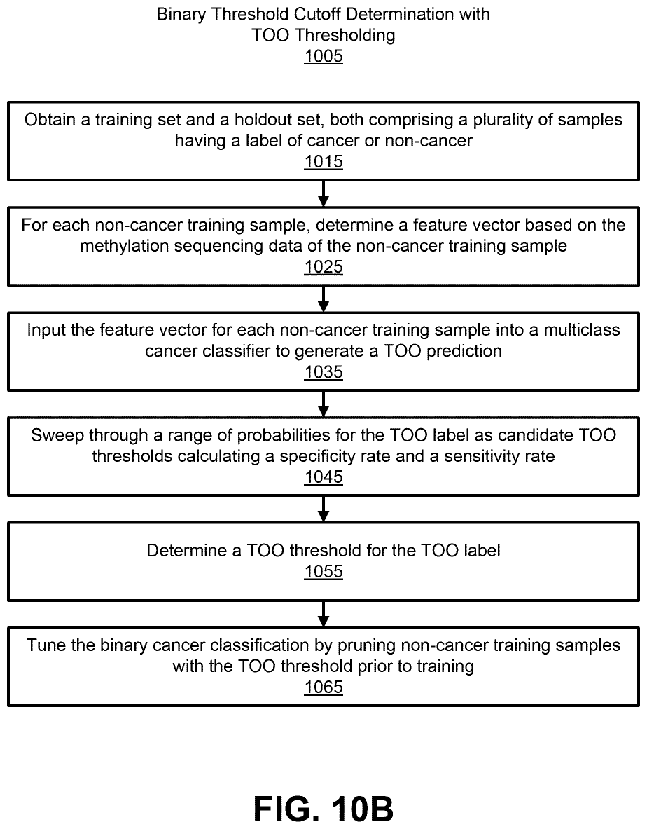

FIG. 10A illustrates a flowchart describing a process 1000 of determining a binary threshold cutoff for binary cancer classification, in accordance with one or more embodiments. A binary classification for predicting between cancer and non-cancer evaluates a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier evaluates a sample's methylation signal (and/or other sequencing data) to determine probabilities for a number of TOO labels classified by the multiclass cancer classifier. A TOO label used in a multiclass cancer classifier can be a cancer tissue type or a cancer tissue subtype (e.g., the hematological subtypes described above). The process 1000 can be performed or accomplished by the analytics system.

The analytics system receives 1010 sequencing data for a plurality of biological samples containing cfDNA fragments, the biological samples comprising cancer samples and non-cancer samples. The sequencing data can be methylation sequencing data, SNP sequencing data, another DNA sequencing data, RNA sequencing data, etc.

For each non-cancer sample, the analytics system classifies 1020 the non-cancer sample using a multiclass cancer classifier based on features derived from the sequencing, wherein the multiclass cancer classifier predicts a probability for each of a plurality of TOO labels. The analytics system can generate a feature vector for the non-cancer sample according to step 320 of FIG. 3A, i.e., assigning an anomaly score for each CpG site in consideration based on at least one anomalously methylated cfDNA fragment overlapping that CpG site.

For each non-cancer sample, the analytics system determines 1030, for one or more TOO labels, whether the predicted probability likelihood exceeds a TOO threshold. The TOO threshold determination is further described below in FIG. 10B.

The analytics system determines 1040 a binary threshold cutoff for predicting a presence of cancer, the binary threshold cutoff determined based on a distribution of non-cancer samples excluding one or more non-cancer samples identified as having a probability likelihood that exceeds at least one TOO threshold. Non-cancer samples, that have at least one probability likelihood for a TOO label that exceeds the TOO threshold corresponding to that TOO label, are excluded. The analytics system then calculates a distribution of the non-cancer samples according to a cancer score for each non-cancer sample and then from the distribution determines the binary threshold cutoff at a desired specificity level (e.g., 99.4-99.9% specificity). It is noted that each cancer score can be determined according to the sequencing data, e.g., the cancer score can be output by a binary cancer classifier predicting a likelihood of cancer based on methylation sequencing data, as described in FIG. 3A. In other embodiments, the cancer score can be calculated according to other methods that input at least sequencing data (e.g., methylation, single nucleotide polymorphism (SNP), DNA, RNA, etc.) and output a sample's likelihood of having cancer based on the input sequencing data.

FIG. 10B illustrates a flowchart describing a process 1005 of thresholding a TOO label for determining a binary threshold cutoff for binary cancer classification, in accordance with one or more embodiments. This process 1005 can be an embodiment of the process 1000. A binary classification for predicting between cancer and non-cancer evaluates a sample's cancer score against a determined binary threshold cutoff, wherein a sample with a cancer score below the binary threshold cutoff is determined to be non-cancer and with a cancer score at or above the binary threshold cutoff is determined to be cancer. A trained multiclass cancer classifier evaluates a sample's methylation signal (and/or other sequencing data) to determine probabilities for a number of TOO labels classified by the multiclass cancer classifier. A TOO label can be a cancer tissue type or more particularly a cancer tissue subtype (e.g., the hematological subtypes described above). The process 1005 can be performed or accomplished by the analytics system.

The analytics system obtains 1015 a training set comprising a plurality of samples having a label of cancer or non-cancer and a holdout set comprising a plurality of samples having a label of cancer or non-cancer, i.e., either a cancer sample or a non-cancer sample, respectively. Each sample in the training set comprises methylation sequencing data, e.g., generated according to the process 100 of FIG. 1A. In other embodiments, each training sample has other sequencing data used in tandem or in substitution of the methylation sequencing data. Moreover, each sample from the training set and the holdout set has a cancer score. As noted above, the cancer score can be determined by the binary classifier as a likelihood that a sample has cancer given the sample's methylation sequencing data. In other embodiments, the cancer score is calculated according to other methods that input at least sequencing data (e.g., methylation, single nucleotide polymorphism (SNP), DNA, RNA, etc.) and output a sample's likelihood of having cancer according to the input sequencing data.

The analytics system, for each non-cancer training sample, determines 1025 a feature vector based on the methylation sequencing data. The analytics system can determine the feature vector for each non-cancer training sample, e.g., in a similar manner to step 320 in FIG. 3A which describes determining an anomaly score for each CpG site in a set of CpG sites considered. In some embodiments, the analytics system defines the anomaly score for the feature vector with a binary score based on whether there is an anomalous fragment in the set of anomalous fragments that encompasses the CpG site. Once all anomaly scores are determined for a sample, the analytics system determines the feature vector as a vector of the anomaly scores associated with each CpG site considered. The analytics system can additionally normalize the anomaly scores of the feature vector based on a coverage of the sample.

The analytics system inputs 1035 the feature vector for each non-cancer training sample into a multiclass cancer classifier to generate a TOO prediction. The multiclass cancer classifier is trained on a plurality of TOO labels, including cancer types, cancer subtypes, non-cancer, or any combination thereof. The multiclass cancer classifier can be trained according to the process 300 of FIG. 3A. The trained multiclass cancer classifier determines, as the cancer prediction, a plurality of probabilities for the TOO labels, wherein a probability for a TOO label indicates likelihood of having a cancer corresponding to the TOO label.

In some examples, the analytics system sweeps 1045 or iterates through a range of probabilities for the TOO label as candidate TOO thresholds calculating a specificity rate and a sensitivity rate over the range of probabilities for the TOO label. The analytics system can sweep through the range of probabilities incrementally, e.g., by 0.01, 0.02, 0.03, 0.04, 0.05, etc. As the analytics system sweeps through the range of probabilities, the analytics system filters non-cancer training samples having a probability of the TOO label at or above the candidate TOO threshold, according to the output of the multiclass cancer classifier. As a numerical example, the analytics system considers a candidate TOO threshold of 0.35. Non-cancer training samples with a probability of the TOO label at or above 0.35 are filtered out of the training set. The analytic system determines an adjusted binary threshold cutoff based on the filtered training set. The analytics system calculates a specificity rate of prediction with the adjusted binary threshold cutoff against the holdout set. The specificity refers to an accuracy of identifying non-Atty. cancer samples as the non-cancer label. The analytics system also calculates a sensitivity rate of prediction with the adjusted binary threshold cutoff against the holdout set. The sensitivity refers to an accuracy of identifying cancer samples as the cancer label. In practice, the specificity rate and/or the sensitivity rate may be defined according to a true positive rate, a false positive rate, a true negative rate, a false negative rate, another statistical calculation, etc.

The analytics system determines 1055 a TOO threshold for the TOO label. The analytics system selects the TOO threshold from the candidate TOO thresholds by optimizing the calculated specificity rates and/or sensitivity rates over the range of candidate TOO thresholds. In some examples, TOO thresholds are determined or otherwise applied for certain TOO tissue type classes or subtype classes, such as hematological classes. Merely by way of example, an algorithm for computing and applying TOO-specific probability thresholds can be used to remove non-cancer samples with exceeding signals of blood disorders. The algorithm can include, for each pre-specified TOO labels, first searching through a grid of probability values, and for every value, evaluating the clinical specificity and the clinical sensitivity of a holdout set using the binary detection threshold computed after removing non-cancer samples with equal or greater probability of the specified TOO label. By iterating through the probability grids, the algorithm will identify a combination of TOO threshold values for the pre-specified TOO labels that optimizes the tradeoff between the clinical specificity and the clinical sensitivity of the holdout set. The final optimized TOO probability threshold values will be used to filter out non-cancer samples that exceeds any of the values given the TOO labels. The cleaned set of non-cancer samples will be used to compute cancer-non-cancer detection threshold. Still, in some examples, the TOO-specific thresholding can be manually set at any cutpoint, such as a desired specificity level (e.g., 99.4-99.9% specificity).

The analytics system tunes 1065 the binary cancer classification by pruning non-cancer training samples exceeding the TOO thresholding prior to determining the binary threshold cutoff. The analytics system filters out non-cancer training samples from the training set according to the determined TOO threshold for the TOO label. The analytics system sets the binary threshold cutoff according to the filtered training set. For example, the analytics system determines a new binary threshold cutoff based on a filtered distribution of scores. In additional embodiments, the analytics system can determine a TOO threshold for any of the TOO labels according to steps 1010, 1020, 1030, and 1040, to tune the binary cancer classification.

### III.C.II. Stratification of Sample Distribution According to TOO Signal

In one or more embodiments, the analytics system tunes the cancer classifier by stratifying the sample distribution according to TOO signal to determine a binary threshold cutoff for each stratum. The analytics system may stratify the sample distribution according to the signal for one or more TOO labels, determined according a TOO prediction output by the multiclass cancer classifier.

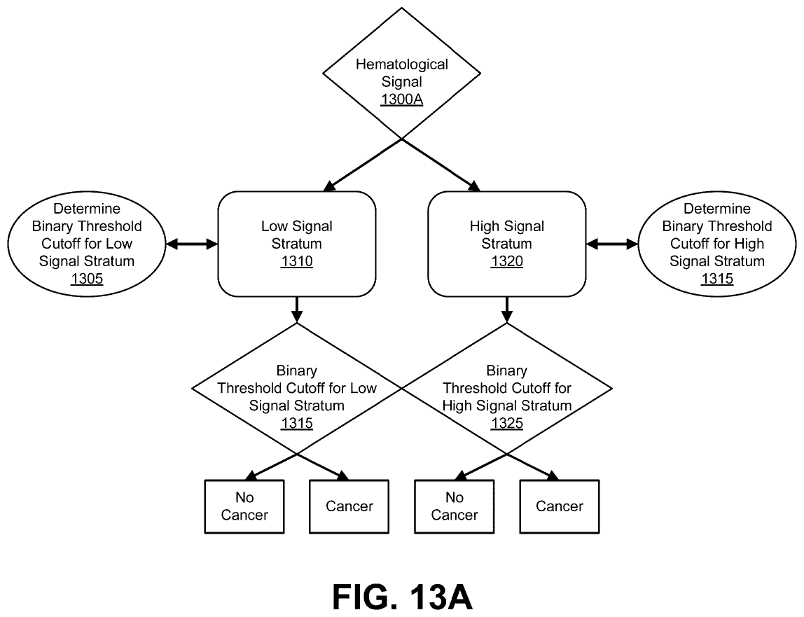

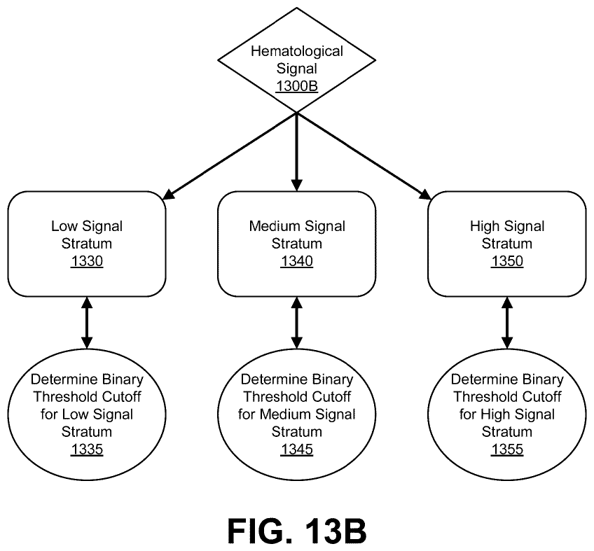

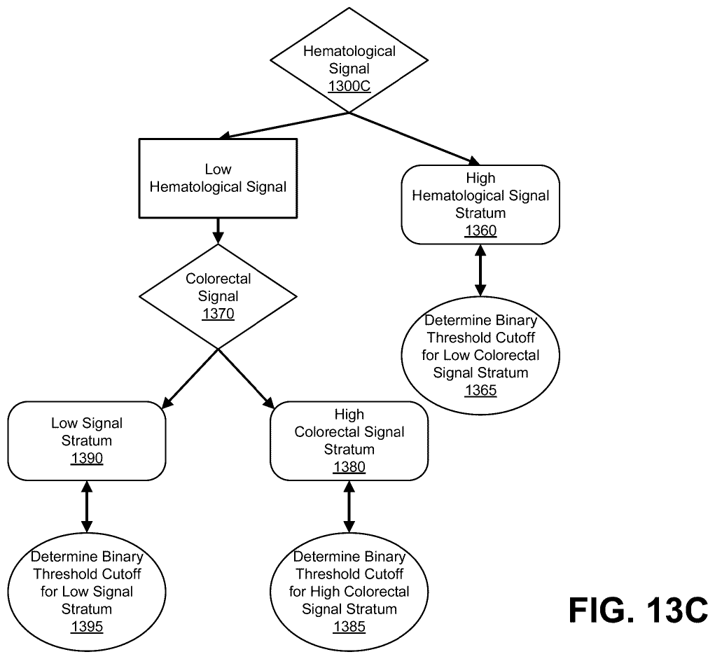

Referring now to FIG. 13A, FIG. 13A illustrates a process for stratifying hematological signals into two strata, in accordance with one or more embodiments. Although the following description describes stratification with a hematological signal, the principles may be readily applied to other TOO signals.

The analytics system stratifies 1300A a holdout set of cancer and non-cancer samples according to the hematological signal into a low signal stratum 1310 and a high signal stratum 1320. Each sample of the holdout set has a cancer score determined by a binary cancer classifier and a TOO prediction determined by a multiclass cancer classifier. In one embodiment, hematological signal for a sample is determined according to a TOO prediction output by a multiclass cancer classifier. High tissue signal may be determined as described under Section III.C. Tuning of Cancer Classifier. In one embodiment, when considering one or more top predictions (e.g., top one, top two, etc.), high hematological signal is determined if at least one of the top predictions being considered is one of a hematological subtype (e.g., lymphoid neoplasm subtype and myeloid neoplasm subtype). Other hematological subtypes may be included. As such, if a sample has a TOO prediction with at least one of the top predictions being considered as the lymphoid neoplasm subtype or the myeloid neoplasm subtype, then the sample is determined to have high hematological signal. Otherwise, the sample is determined not to have high hematological signal.

The analytics system determines a binary threshold cutoff for each stratum for predicting presence or absence of cancer of a sample. The samples in the low signal stratum 1310 are used by the analytics system to determine 1305 a binary threshold cutoff for predicting absence or presence of cancer in samples in the low signal stratum 1310. The binary threshold cutoff is determined 1305 according to a false positive budget set for the low signal stratum 1310. With cancer scores for the samples in the low signal stratum 1310, the analytics system sweeps through a range of candidate binary threshold cutoffs evaluating a true positive rate (also referred to as sensitivity) and a false positive rate at each candidate binary threshold cutoff. The candidate binary threshold cutoff with a false positive rate that is closest within the false positive budget is determined to be the candidate binary threshold cutoff. The analytics system performs similar operations to determine 1315 a binary threshold cutoff for the high signal stratum 1320. The false positive budget for the low signal stratum 1310 and the false positive budget for the high signal stratum 1320 may be set according to a ratio of statistical true positive rates of the strata. The ratio aims to suppress the false positive rate in the high signal stratum 1320.

For a test sample, the analytics system places the test sample into either the low signal stratum 1310 or the high signal stratum 1320 according to hematological signal. If the test sample is placed in the low signal stratum 1310, then the analytics system applies 1315 the binary threshold cutoff for the low signal stratum 1310 to the cancer score of the test sample. If the cancer score is greater than or equal to the binary threshold cutoff for the low signal stratum 1310, then the analytics system returns a prediction of cancer presence in the test sample, and returns a prediction of no cancer otherwise. If test sample is placed in the high signal stratum 1320, then the binary threshold cutoff for the low signal stratum 1320 is applied 1325 to the cancer score of the test sample. If the cancer score is greater than or equal to the binary threshold cutoff for the high signal stratum 1320, then the analytics system returns a prediction of cancer presence in the test sample, and returns a prediction of no cancer otherwise.

FIG. 13B illustrates a process for stratifying hematological signals into three strata, in accordance with one or more embodiments. Although the following description describes stratification with a hematological signal, the principles may be readily applied to other TOO signals. The principles may also be readily extended to stratification into numbers of strata beyond three.

The analytics system stratifies a holdout set of cancer and non-cancer samples into three strata according to hematological signal: a low signal stratum 1330, a medium signal stratum 1340, and a high signal stratum 1350. Each sample of the holdout set has a cancer score determined by a binary cancer classifier and a TOO prediction determined by a multiclass cancer classifier. In one or more embodiments, a hematological TOO label comprises multiple hematological subtypes. Any sample of the holdout set with a high tissue signal in one or more aggressive hematological subtypes is placed into the high signal stratum 1350. Any sample of the holdout set (not already classified into the high signal stratum 1350) with a high tissue signal in one or more indolent hematological subtypes is placed into the medium signal stratum 1340. And samples not classified in either the high signal stratum 1350 or the medium signal stratum 1340 are placed into the low signal stratum 1330. According to the principles described in FIG. 13A, the analytics system determines a binary threshold cutoff for each stratum based on a false positive budget for each stratum-a binary threshold cutoff for the low signal stratum 1330 is determined 1335, a binary threshold cutoff for the medium signal stratum 1340 is determined 1345, and a binary threshold cutoff for the high signal stratum 1350 is determined 1355. Similar to FIG. 13A, for a test sample, the analytics system identifies a stratum in which to place the test sample and applies the binary threshold cutoff for that stratum to predict the presence or absence of cancer in the test sample.

FIG. 13C illustrates a process for first stratifying hematological signals, and subsequently stratifying colorectal signals, in accordance with one or more embodiments. Although the following description describes stratification with a hematological signal and subsequently with a colorectal signal, the principles may be readily applied to other combinations of TOO signals serially evaluated.

The analytics system stratifies a holdout set of cancer and non-cancer samples according to hematological signal 1300C and subsequently a colorectal signal 1370. Each sample of the holdout set has a cancer score determined by a binary cancer classifier and a TOO prediction determined by a multiclass cancer classifier. Similar to the principles described above in FIG. 13A, any sample of the holdout set having a high hematological signal is placed into a high hematological signal stratum 1360. Remaining samples are subsequently stratified according to colorectal signal 1370. Analogous to the hematological stratification, any sample with a high colorectal signal is placed into a high colorectal signal stratum 1380. Samples placed into neither the high hematological signal stratum 1360 nor the high colorectal signal stratum 1380 are grouped into the low signal stratum 1390. In this embodiment, the hematological signal is of a higher priority than the colorectal signal. In one or more embodiments, a plurality of TOO signals may be serially evaluated in order of priority. As such, a sample having both high hematological signal and high colorectal signal would be placed under the high hematological stratum 1360 and not under the high colorectal stratum 1380, as the hematological signal is of higher priority than the colorectal signal. According to the principles described in FIG. 13A, the analytics system determines a binary threshold cutoff for each stratum based on a false positive budget for each stratum. A binary threshold cutoff for the high hematological signal stratum 1360 is determined 1365, a binary threshold cutoff for the high colorectal signal stratum 1380 is determined 1385, and a binary threshold cutoff for the low signal stratum 1390 is determined 1395. Similar to FIG. 13A, for a test sample, the analytics system identifies a stratum in which to place the test sample and applies the binary threshold cutoff for that stratum to predict the presence or absence of cancer in the test sample.

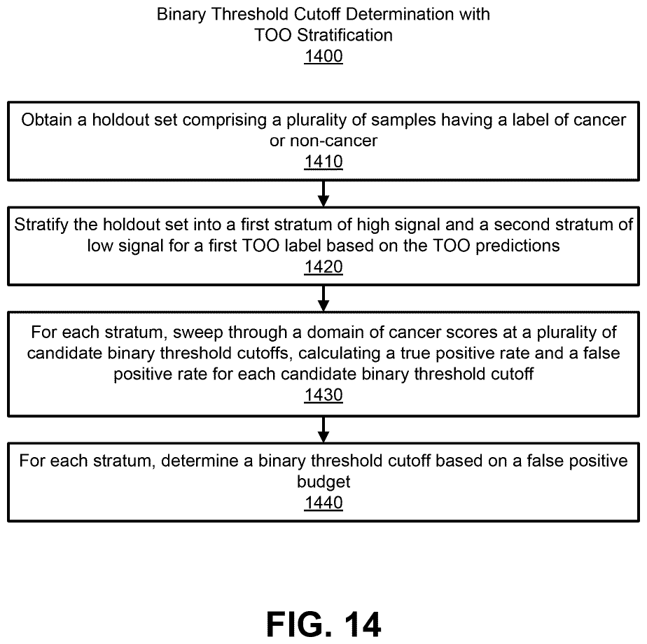

FIG. 14 illustrates a process 1400 of determining binary threshold cutoffs for TOO stratification, in accordance with one or more embodiments. Although the process 1400 is described as being performed by the analytics system, the process 1400 may more generally be performed by any computing system.

The analytics system obtains 1410 a holdout set comprising a plurality of samples classified as or having a label of cancer or non-cancer. Each sample of the holdout set is accompanied with a cancer score, for instance, representative of a likelihood that the sample corresponds to cancer (e.g., determined by a binary cancer classifier), and a TOO prediction, for instance representative of a likelihood that the sample corresponds to cancer of a particular type of tissue (e.g., determined by a multiclass cancer classifier).

The analytics system stratifies 1420 the holdout set into a first stratum of high signal and a second stratum of low signal for a first TOO label based on the TOO predictions. In one example, the stratification uses a prediction value threshold. Any sample with a prediction value for the first TOO label in the TOO prediction at or above the prediction value threshold is classified as high signal for the first TOO label. Otherwise, the sample is classified as low signal for the first TOO label. In another example, the analytics system considers one or more top predictions in a TOO prediction for each sample. Any sample with the first TOO label in at least one of the top predictions being considered is classified as high signal for the first TOO label. Otherwise, the sample is classified as low signal for the first TOO label.

In one or more embodiments, the analytics system further stratifies into a third stratum of medium signal for the first TOO label. In embodiments where signal is stratified according to prediction value, the range of prediction values may be segmented into three portions for determining high signal, medium signal, and low signal.

In one or more embodiments, the analytics system further stratifies one or more strata into additional strata according to tissue signal for one or more additional TOO labels. The additional TOO labels may be a lower priority in stratification than the first TOO label.

The analytics system, for each stratum, sweeps 1440 through a domain of cancer scores at a plurality of candidate binary threshold cutoffs, calculating a true positive rate and a false positive for each candidate binary threshold cutoff. The true positive rate can be plotted against the false positive rate to generate a receiver operator characteristic (ROC) curve.

The analytics system, for each stratum, determines 1440 a binary threshold cutoff based on a false positive budget. The false positive budget may be allocated to each stratum according to a ratio of statistical true positive rates of the strata.

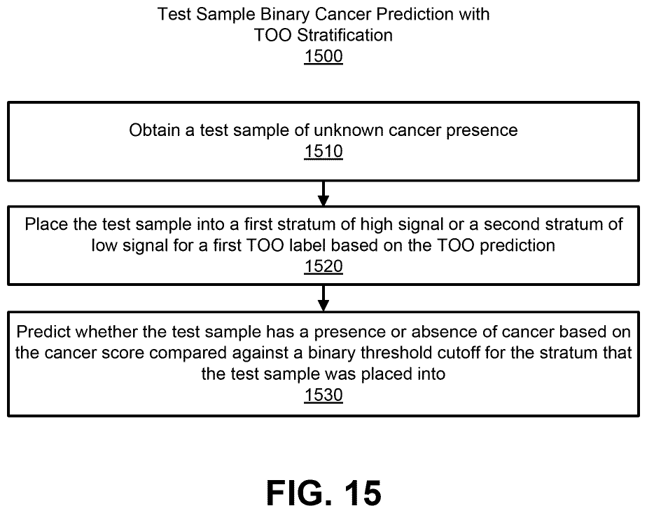

FIG. 15 illustrates a flowchart describing a process 1500 of predicting cancer presence or cancer absence for a test sample using a binary threshold cutoff determined by TOO stratification, in accordance with one or more embodiments. The process 1500 is described as being performed by the analytics system, though the process 1500 may more generally by accomplished by any computing system.

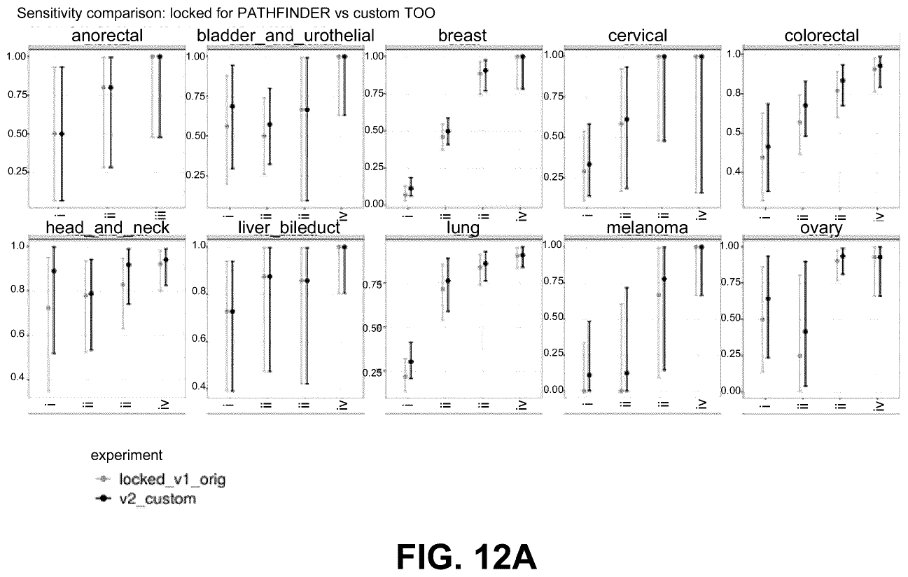

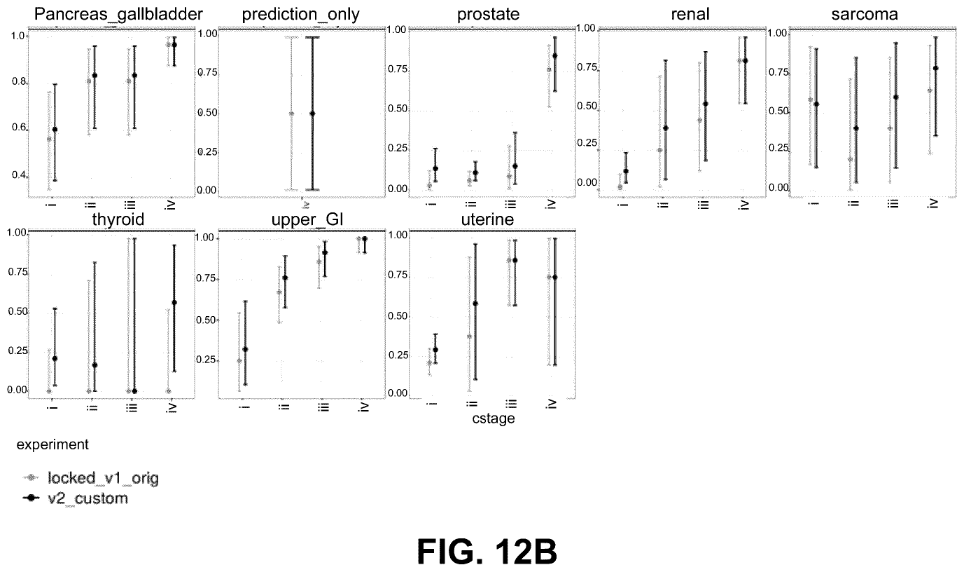

The steps of FIG. 15 form the second stage of the hierarchical architecture introduced with reference to FIGS. 12A-12B. Each step is executed on features vector that reduce computational requirements while preserving specific methylation signal. By stratifying tissue samples and applying a stratum-specific detector whose binary cutoff has been determined against a predefined false-positive budget, the process converts a high-dimensional, class-imbalanced classification task into two smaller, balanced sub-tasks. As noted in the data figures herein, this routing lowers the false-positive rate for low-signal tissues without degrading sensitivity for high-signal tissues, demonstrating an improvement in machine-learning performance that manifests directly in diagnostic accuracy.

The ordered combination of steps (e.g., confidence-based stratification, threshold selection constrained by the budget, and inference on the features vectors) implements a specific sequence of data-transformations that conventional single-stage pipelines do not perform. As each transformation manages input properties of features presented to the binary detector and relies on parameters derived through the threshold optimizer, the method achieves more than a generic application of machine learning because it realizes a computational workflow that both improves model calibration and reduces resource consumption.

To elaborate, the analytics system obtains 1510 a test sample of unknown cancer presence. The test sample is accompanied with a cancer score, e.g., determined by a binary cancer classifier, and a TOO prediction, e.g., determined by a multiclass cancer classifier.

The analytics system places 1520 the test sample into a first stratum of high signal or a second stratum of low signal for a first TOO label based on the TOO prediction. Placement (or classification) is described above (for instance, with regards to stratification at step 1420 of the process 1400).

The analytics system predicts 1530 whether the test sample has a presence or absence of cancer by comparing the cancer score against a binary threshold cutoff for the stratum that the test sample was placed into. For example, if the test sample had high signal for the first TOO label and was placed into the first stratum of high signal, then the analytics system applies the binary threshold cutoff determined for the first stratum of high signal to the cancer score of the test sample. Alternatively, if the test sample was placed into the second stratum of low signal, then the binary threshold cutoff determined for the second stratum is used. If the cancer score of the test sample is at or above the binary threshold cutoff used, then the test sample is predicted to have a presence of cancer. Otherwise, the test sample is predicted to be absent of cancer.

### III.D. Deployment of Cancer Classifier

During use of the cancer classifier, the analytics system obtains a test sample from a subject of unknown cancer type. The analytics system may process the test sample comprised of DNA molecules with any combination of the processes 100, 200, and 220 to achieve a set of anomalous fragments. The analytics system determines a test feature vector for use by the cancer classifier according to similar principles discussed in the process 300. The analytics system calculates an anomaly score for each CpG site in a plurality of CpG sites in use by the cancer classifier. For example, the cancer classifier receives as input feature vectors inclusive of anomaly scores for 1,000 selected CpG sites. The analytics system thus determines a test feature vector inclusive of anomaly scores for the 1,000 selected CpG sites based on the set of anomalous fragments. The analytics system calculates the anomaly scores in a same manner as the training samples. In one embodiment, the analytics system defines the anomaly score as a binary score based on whether there is a hypermethylated or hypomethylated fragment in the set of anomalous fragments that encompasses the CpG site.

The analytics system then inputs the test feature vector into the cancer classifier. The function of the cancer classifier then generates a cancer prediction based on the classification parameters trained in the process 300 and the test feature vector. In the first manner, the cancer prediction is binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 360 of the process 300 with another cancer classifier trained in step 370 or the process 300. The analytics system inputs the test feature vector into the cancer classifier trained as a binary classifier in step 360 of the process 300. The analytics system receives an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier receives the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system determines a cancer score for a test sample based on the test sample's sequencing data (e.g., methylation sequencing data, SNP sequencing data, other DNA sequencing data, RNA sequencing data, etc.). The analytics system compares the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

### IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

### IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V. Example Results of Cancer Classifier

### V.A. Sample Collection and Processing

Study design and samples: CCGA (NCT02889978) is a prospective, multi-center, case-control, observational study with longitudinal follow-up. De-identified biospecimens were collected from approximately 15,000 participants from 142 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, MD). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, MI) and constructed libraries were quantified using KAPA Library Quantification Kit for Illumina Platforms (Kapa Biosystems; Wilmington, MA). Four libraries along with 10% PhiX v3 library (Illumina, FC-110-3001) were pooled and clustered on an Illumina NovaSeq 6000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.B. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypo-methylated). This procedure resulted in a median (range) of 2,800 (1,500-12,000) UFXM fragments for participants without cancer in training, and a median (range) of 3,000 (1,200-220,000) UFXM fragments for participants with cancer in training. As this data reduction procedure only used reference set data, this stage was only required to be applied to each sample once.

### V.B. Tissue of Origin Cancer Classification

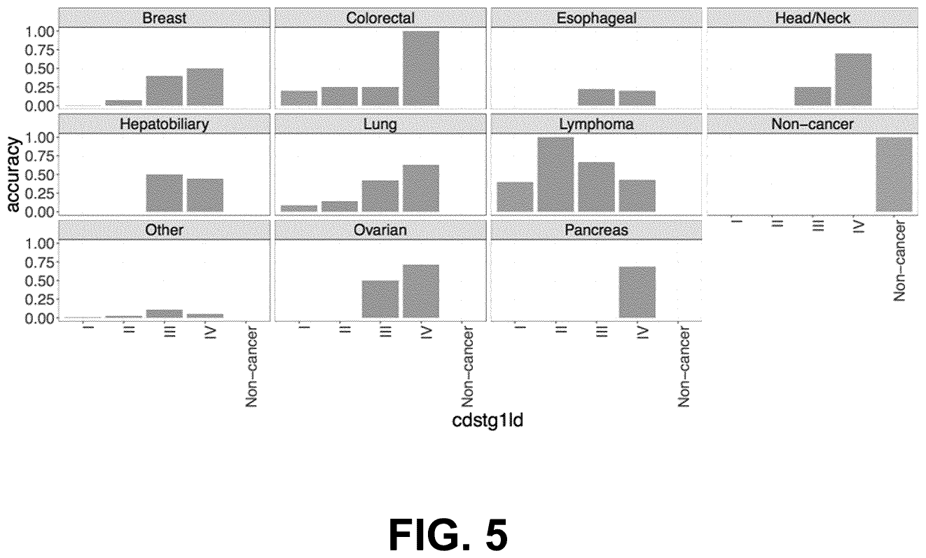

FIGS. 5-7, 11, 12A, 12B, 16A, 16B, 17, and 18 illustrate many graphs showing cancer prediction accuracy of various trained cancer classifiers, according to an embodiment. The cancer classifiers used to produce results shown in FIGS. 5-7, 11, 12A, 12B, 16A, 16B, 17, and 18 are trained according to example implementations of the process 300 described above in FIG. 3A.

The analytics system selects CpG sites to be considered in the cancer classifier. The information gain is computed for training samples with a given cancer type compared to all other samples. For example, two random variables ‘anomalous fragment’ (‘AF’) and ‘cancer type’ (‘CT’) are used. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. For a given cancer type, the analytics system uses this information to rank CpG sites based on how cancer specific they are. This procedure is repeated for all cancer types under consideration. The ranked CpG sites for each cancer type are greedily added (e.g., to achieve approximately 3,000 CpG sites) for use in the cancer classifier.

For featurization of samples, the analytics system identifies fragments in each sample with anomalous methylation patterns and furthermore UFXM fragments. For one sample, the analytics system calculates an anomaly score for each selected CpG site for consideration (˜3,000). The analytics system defines the anomaly score with a binary scoring based on whether the sample has a UFXM fragment that encompasses the CpG site.

FIG. 5 illustrates many graphs showing cancer prediction accuracy of a multiclass cancer classifier for various cancer types, according to an example implementation. In this illustrative example, the multiclass cancer classifier is trained to distinguish feature vectors according to 11 cancer types: breast cancer type, colorectal cancer type, esophageal cancer type, head/neck cancer type, hepatobiliary cancer type, lung cancer type, lymphoma cancer type, ovarian cancer type, pancreas cancer type, non-cancer type, and other cancer type. The samples used in this example were from subjects known to have each of the cancer types. For example, a cohort of breast cancer type samples were used to validate the cancer classifier's accuracy in calling the breast cancer type. Moreover, the samples used are from subjects in varying stages of cancer.

For the breast cancer cohort, the colorectal cancer cohort, and the lung cancer cohort, the cancer classifier was gradually more accurate in accurately predicting the cancer type in subsequent stages of cancer. For the head/neck cohort, ovarian cohort, and pancreas cohort, the cancer classifier had accuracy increases in the latter stage, i.e., Stage III and/or Stage IV. For the esophageal cohort and the hepatobiliary cohort, the cancer classifier also had latter stage accuracy, i.e., Stage III and Stage IV. With the non-cancer cohort, the cancer classifier was perfectly accurate in predicting the non-cancer samples to not likely have cancer. Last but not least, the lymphoma cohort had success throughout varying stages with a peak success in accurately predicting samples in Stage II of cancer.

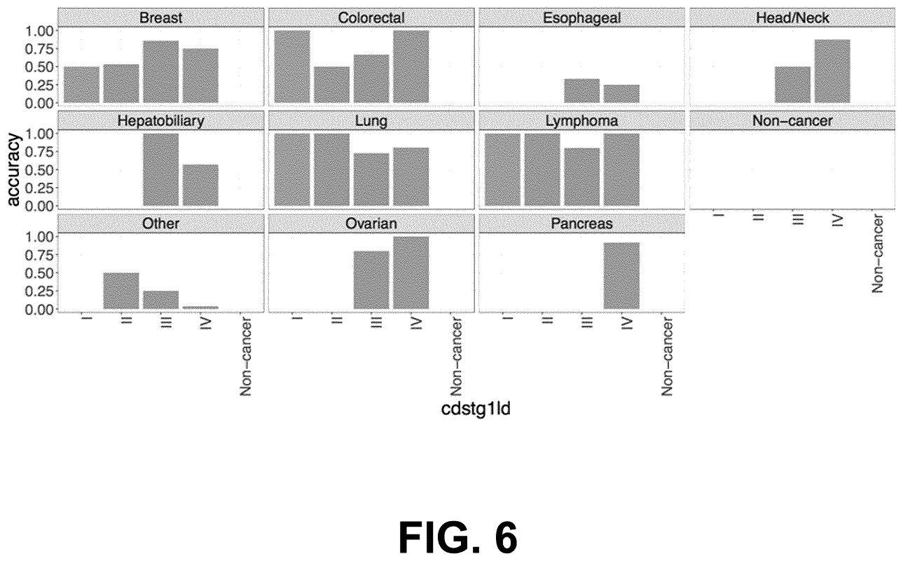

FIG. 6 illustrates many graphs showing cancer prediction accuracy of a multiclass cancer classifier for various cancer types after first using a binary cancer classifier, according to an example implementation. In this example, the analytics system first inputs the samples from many cancer type cohorts into the binary cancer classifier to determine whether or not the samples likely have or do not have cancer. Then the analytics system inputs samples that are determined to likely have cancer into the multiclass cancer classifier to predict a cancer type for those samples. The cancer types in consideration include: breast cancer type, colorectal cancer type, esophageal cancer type, head/neck cancer type, hepatobiliary cancer type, lung cancer type, lymphoma cancer type, ovarian cancer type, pancreas cancer type, and other cancer type.

In comparison to the example in FIG. 5, the analytics system showed an increase in accuracy when first using the binary cancer classifier then the multiclass cancer classifier. Among the breast cancer cohort, the colorectal cancer cohort, the lung cancer cohort, and the lymphoma cancer cohort, the analytics system had overall increases in accuracy. In particular, the analytics system had stark increases in prediction accuracy for each of those cancer types in early stages of cancer, i.e., Stage I, Stage II, and even Stage III.

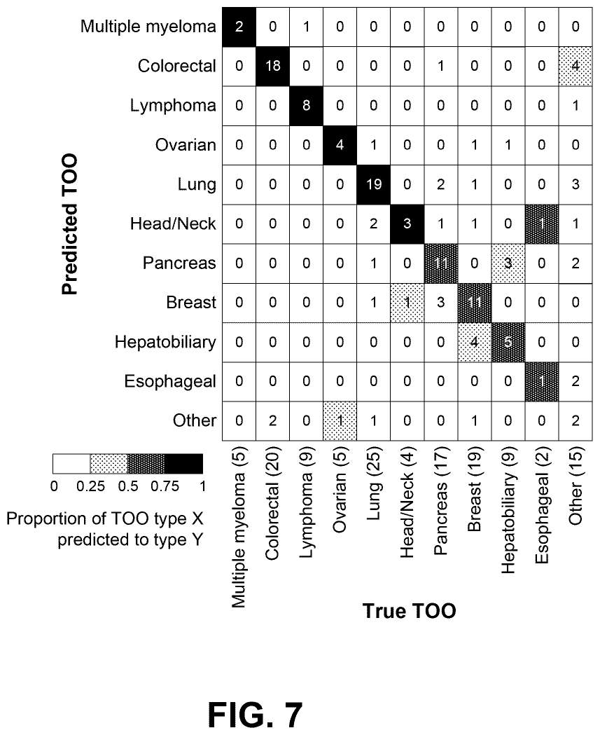

FIG. 7 illustrates a confusion matrix demonstrating performance of a trained cancer classifier, according to an example implementation. In one example of training according to the process 300, a multiclass kernel logistic regression (KLR) classifier with ridge regression penalty was trained on the derived feature vectors with a penalty on the weights, and a fixed penalty on the bias term for each cancer type. The ridge regression penalty was optimized on a portion of the training data not used in selecting high-relevance locations (using log-loss), and, once the optimum parameter was found, the logistic classifier was retrained on the whole set of local training folds. The selected high-relevance sites and classifier weights were then applied to new data. Within the CCGA training set, one fold was repeatedly held out, relevant sites on 8 of the 9 folds were selected, the hyper-parameters for the KLR classifier were optimized on the 9th set, and the KLR was retrained on 9 of 10 folds and applied to the held-out fold. This was repeated 10 times to estimate TOO within the CCGA training set. For the CCGA test set, relevant sites were selected on 9/10 folds of CCGA train, hyper-parameters were optimized on the 10th fold, and the KLR classifier was retrained on all CCGA training data and the selected sites and the KLR classifier were applied to the test set. The cancer types considered include: multiple myeloma cancer type, colorectal cancer type, lymphoma cancer type, ovarian cancer type, lung head/neck cancer type, pancreas cancer type, breast cancer type, hepatobiliary cancer type, esophageal cancer type, and other cancer type. Other cancer type included cancers with less than 5 samples collected within CCGA, such as anorectal, bladder, cancer of unknown primary TOO, cervical, gastric, leukemia, melanoma, prostate, renal thyroid, uterine, and other additional cancers.

The confusion matrix shows agreement between cancer types having samples with known cancer TOO (along x-axis) and predicted cancer TOO (along y-axis). To validate performance of the trained KLR classifier, a cohort of samples (indicated in parentheses along the y-axis for each cancer type) for each cancer type was classified with the KLR classifier. The x-axis indicates how many samples from each cohort was classified under each cancer type. For example, with the lung cancer cohort having 25 samples with known lung cancer, the KLR classifier predicted one sample to have ovarian cancer, nineteen samples to have lung cancer, two samples to have head/neck cancer, one sample to have pancreas cancer, one sample to have breast cancer, and one sample to be labeled as other cancer type. Notably, for all cancer types except other cancer type, the KLR classifier accurately predicted more than half of each cohort with particularly high accuracy for the cancer types of multiple myeloma ( 2/2 or 100%), colorectal ( 18/20 or 90%), lymphoma ( 8/9 or 88.8%), ovarian (⅘ or 80%), lung ( 19/25 or 76%), and head/neck (¾ or 75%). These results demonstrate the predictive accuracy of the KLR classifier.

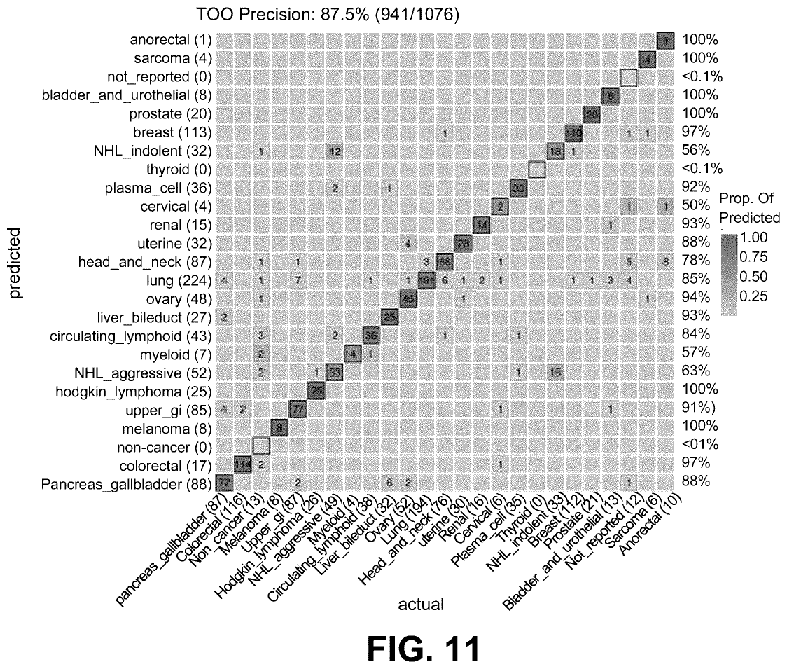

FIG. 11 illustrates a confusion matrix demonstrating performance of a trained cancer classifier with additional hematological cancer subtypes, in an example implementation. The cancer classifier may be trained according to the principles described above, for instance with regards to the cancer classifier example results of FIG. 7. The TOO labels for hematological subtypes include Hodgkin's-Lymphoma (HL), NHL aggressive, NHL indolent, myeloid, circulating lymphoma (or lymphoid), and plasma cell. Of note, the classification precision is 87.5% over 1,076.

FIGS. 12A and 12B illustrate graphs showing cancer prediction accuracy for numerous cancer types over stages of cancer, in an example implementation. In this example, the cancer classifier is trained after pruning the non-cancer samples according to the process 1000 described above. The analytics system determined multiple TOO thresholds for the hematological subtypes. The analytics system excluded non-cancer samples with at least one TOO probability at or above the corresponding TOO threshold for the hematological subtypes. The graphs shown show the classification sensitivity over varying stages of cancer for cancer types: anorectal, bladder and urothelial, breast, cervical, colorectal, head and neck, liver and bile duct, lung, melanoma, ovary, pancreas and gallbladder, prostate, renal, sarcoma, thyroid, upper gastrointestinal, and uterine. A graph for each cancer type shows the prediction sensitivity over each stage of the cancer type with a first cancer classifier without TOO thresholding labeled as “locked_v1_orgi” and a second cancer classifier with TOO thresholding labeled as “v2_custom”. Notably, for many cancer types the second cancer classifier has higher prediction accuracy while maintaining a tight confidence interval, given more samples available for validation. Of particular note, there are higher prediction accuracies in many cancer types at the stage I and II levels, indicating improved prediction potential with TOO thresholding in early stage cancers. Example Hematologic-Specific Classifier

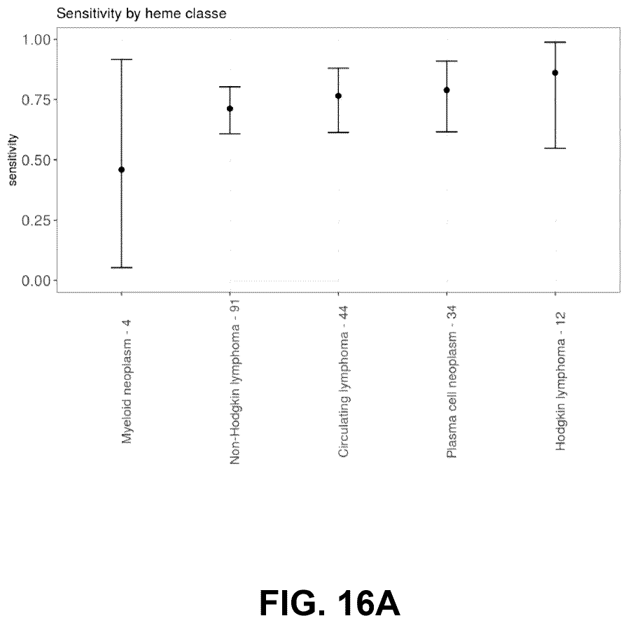

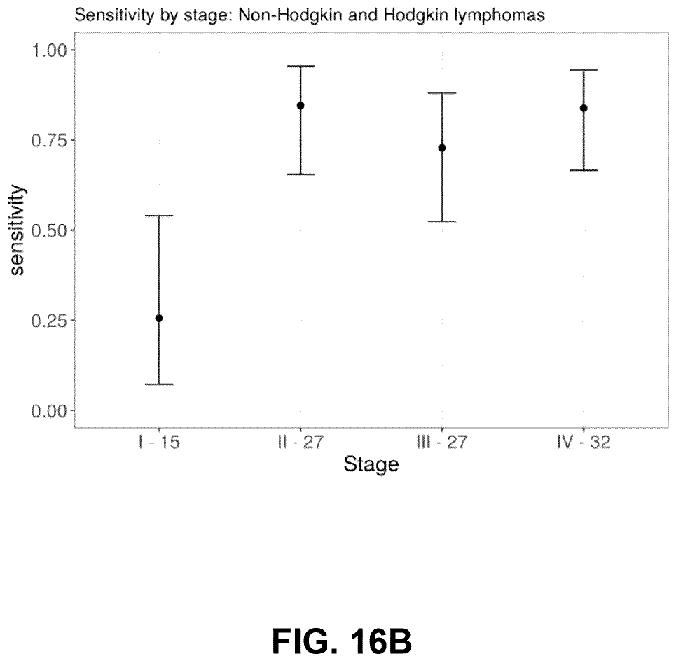

FIGS. 16A, 16B, 17, and 18 illustrate graphs showing cancer prediction accuracy for a hematological-specific cancer classifier, according to a first example implementation. First, cfDNA samples were accessed from a second pre-specified sub-study of CCGA, which was designed for targeted methylation assay validation. In this example, only training set samples were used, and tumor tissue samples from an in-house tissue biopsy reference database were included for the classification model training. Specifically, the samples used to train the custom classification model for hematological malignancies were from participants enrolled with a hematological cancer diagnosis (cancer cases) and participants enrolled without a cancer diagnosis (non-cancer controls). 154 blood cell samples or tissue FFPE samples of hematological malignancies were also included. In total, cfDNA samples from 185 participants with hematological cancers and 1,998 non-cancer controls confirmed without cancer diagnosis at the one year follow-up were included for performance evaluation.

In this first example implementation, the cancer classifier is trained to distinguish between five hematological subtypes and an absence of cancer (“non-cancer”). The five hematological subtypes are myeloid neoplasm, non-Hodgkin lymphoma (NHL), circulating lymphoma, plasma cell neoplasm, and Hodgkin lymphoma (HL). A cross-validated mutual information-based algorithm was used to identify features that discriminated between the five hematological subtypes and the control class. A multinomial classifier was then trained to detect the presence or absence of cancer and predict tissue of origin among the five hematological cancers and non-cancers using 6-fold cross-validation.

The trained hematological-specific classifier achieved an overall specificity of >99.4% [95% Confidence Interval (CI): 99.0-99.7%] in controls, and a sensitivity of 74.3% [95% CI: 67.4-80.5%] for hematological cancers. FIG. 16A illustrates a graph showing the classifier's sensitivity at 99.5% specificity level across the hematological subtypes. The sensitivity of the hematological-specific cancer classifier for each hematological subtype is arranged in ascending order, with the number in the class label indicating the number of samples and the error bars showing the 95% confidence intervals. Myeloid neoplasm, having four samples classified, have a sensitivity just below 50% with a wide 95% confidence interval ranging from ˜10% to ˜90%. This lower sensitivity may be due to limited samples used in training. In the embodiment of FIG. 16A, NHL, circulating lymphoma, plasma cell neoplasm, and HL have better sensitivities than myeloid neoplasm, around 70% to 87%. Specifically, the sensitivities by hematological subtypes were 45.8% [95% CI: 5.3-91.6%] for myeloid neoplasms, 76.5% [95% CI: 61.3-88.0%] for circulating lymphomas, 86.1% [95% CI: 54.7-98.7%] for Hodgkin lymphomas, 71.3% [95% CI: 60.8-80.3%] for other Non-Hodgkin lymphomas, and 78.9% [95% CI: 61.6-91.0%] for plasma cell neoplasms.

FIG. 16B illustrates a graph showing the classifier's sensitivity at 95% specificity across stages for Hodgkin lymphomas and Non-Hodgkin lymphomas. Stage I sensitivity (out of 15 samples) is ˜25%. Stage II sensitivity (out of 27 samples) is ˜85%. Stage III sensitivity (out of 27 samples) is ˜75%. Stage IV sensitivity (out of 32 samples) is ˜85%. This graph shows a dramatic increase in sensitivity of the hematological-specific cancer classifier between Stage I and Stage II (and further). Specifically, for lymphomas (HL and NHL), the sensitivities by stages were 25.6% [95% CI: 7.2-54.0%] for stage I, 84.6% [95% CI: 65.5-95.5%] for stage II, 72.8% [95% CI: 52.4-88.0%] for stage III, and 83.9% [95% CI: 66.6-94.4%] for stage IV.

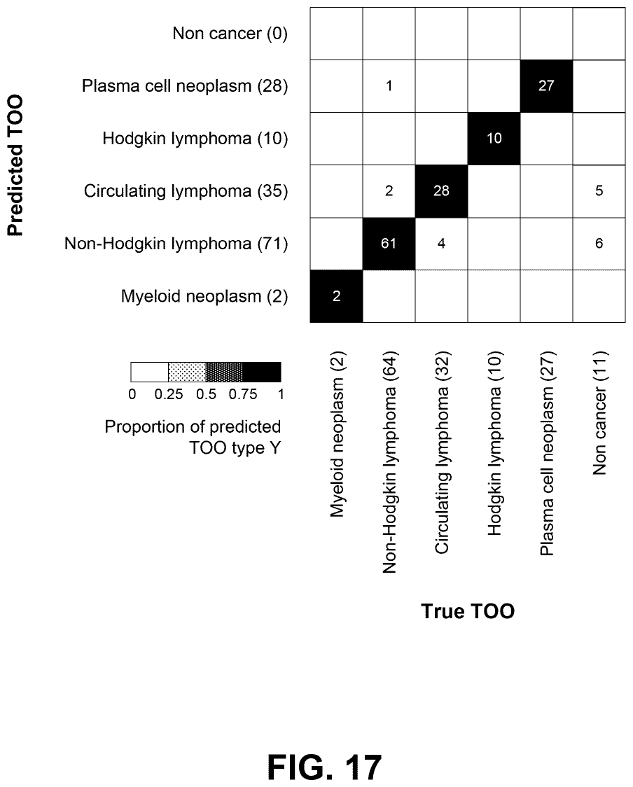

FIG. 17 illustrates a confusion matrix showing cancer prediction accuracy of the hematologic-specific cancer classifier in the first example implementation. The numbers in each box represent the total number of samples predicted. In the confusion matrix, coloring/shading corresponds to the proportion of the predicted hematological subtype, as indicated to the right of the plot. Likewise, the percentage of predictions that are correct is indicated to the right of the graph. The tissue of origin localization was assessed on cancer cases that were correctly detected by a TOO multiclass classifier as hematological cancers. As shown at FIG. 17, the hematological-specific classifier achieved an overall TOO prediction accuracy of 87.7%, with Hodgkin lymphoma and myeloid neoplasms showing the highest prediction accuracy (100%) followed by plasma cell neoplasm (96.4%), Non-Hodgkin lymphoma (85.9%), and circulating lymphomas (80%). Of the 11 non-cancer controls (0.55% of non-cancer controls) who were classified as having hematological cancers, five were predicted as circulating lymphoma, and six were predicted as other Non-Hodgkin lymphoma (<1% false positive rate), most showing confident TOO signal localizing to the predicted heme class (≥50% of total probability mass).

In some examples, a low dimensional representation of the methylation features active for the final classifier can be generated using the UMAP method, which preserves the topology of high dimensional data. For example, at FIG. 9B discussed above, the UMAP embedding shows that the majority of hematological malignancies separated into five major clusters reflecting developmental lineages and disease ontogeny. The vast majority of non-cancer controls (shown using contour density at FIG. 9B) were clustered separate from the hematological cancers.

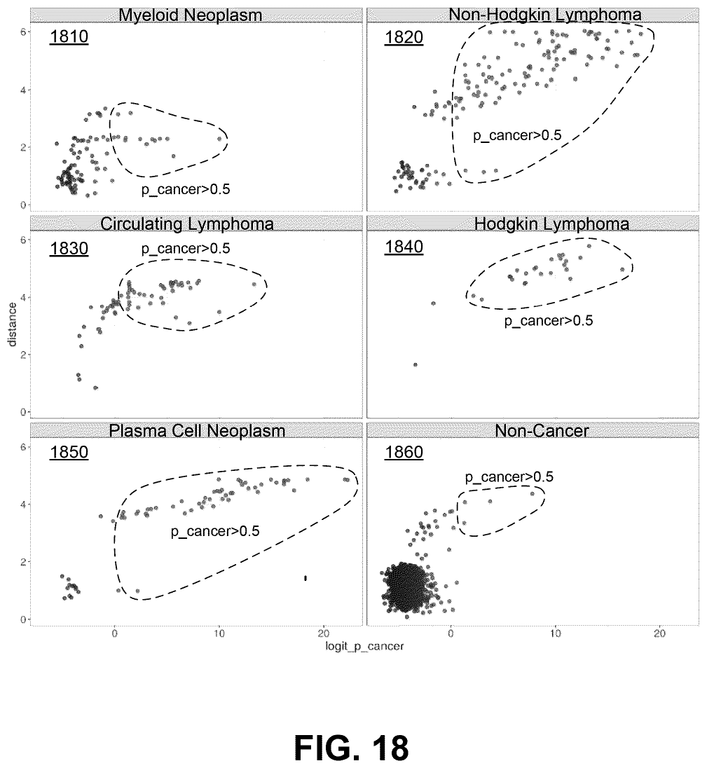

Scatter plots were then used to visualize the relationship between the spatial distribution of feature embeddings and the model's class probabilities. For example, FIG. 18 illustrates a series of graphs plotting cancer score against distance from the centroid in the UMAP embedding, in an example implementation. The UMAP embedding is the same as the UMAP embedding of FIG. 9B. The x-axis plots the logit transformed probability of a sample being cancer—i.e., logit of cancer score. The logit function (also referred to as the log-odds) is the logarithm of the odds p/1-p, where p is probability. The y-axis plots the Euclidean distance from the centroid of the UMAP embedding. These plots depict a correlation between the cancer score and the localization in the UMAP embedding for the various hematological subtypes. Graph 1810 depicts the correlation in the myeloid neoplasm subtype. Graph 1820 depicts the correlation in the NHL subtype. Graph 1830 depicts the correlation in the circulating lymphoma subtype. Graph 1840 depicts the correlation in the HL subtype. Graph 1850 depicts the correlation in the plasma cell neoplasm subtype. Graph 1860 depicts minimal correlation in the non-cancer samples. As shown at FIG. 18, there was a strong positive correlation between their UMAP embedding localization and classification score.

As demonstrated by the example hematological-specific classifier, the custom classifier for hematological malignancies offers a convenient way to simultaneously detect and distinguish five major hematological malignancies, which can facilitate clinical diagnosis and treatment selection. In this way, the custom classifier can achieve even more sensitive detection of multiple cancers and can be used to refine cancer detection and TOO prediction accuracy.

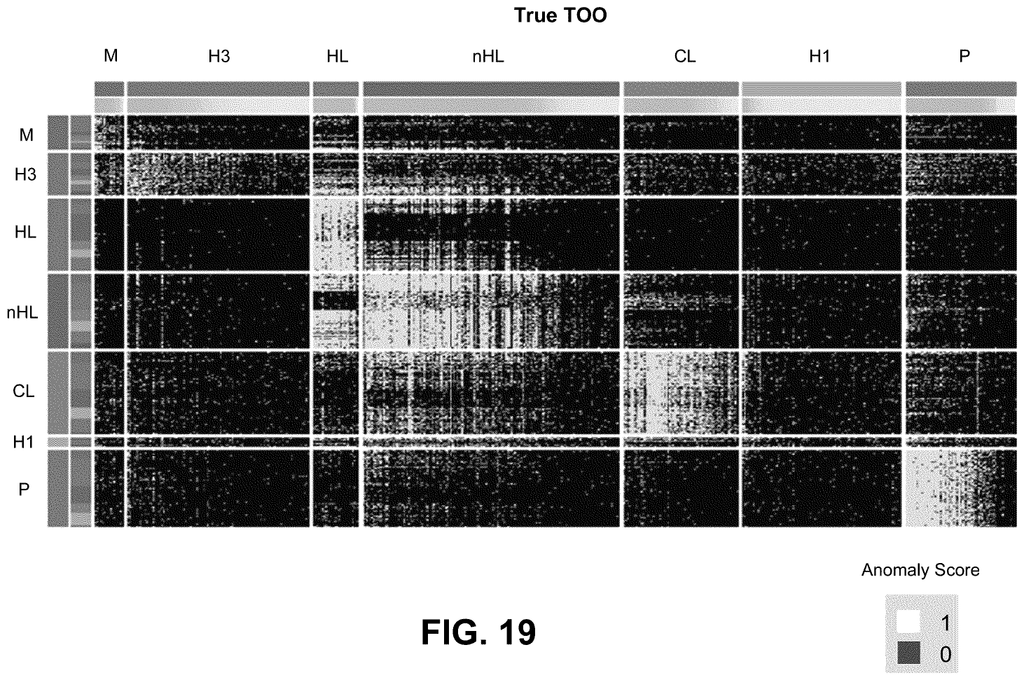

FIGS. 19, 20, and 21 illustrate graphs showing cancer prediction accuracy for a hematological-specific cancer classifier, according to a second example implementation. First, cfDNA samples were accessed from a second pre-specified sub-study of CCGA, which was designed for targeted methylation assay validation. In this example, only training set samples were used, and tumor tissue samples from an in-house tissue biopsy reference database were included for the classification model training. Specifically, the samples used to train the custom classification model for hematological malignancies were from participants enrolled with a hematological cancer diagnosis (cancer cases) and participants enrolled without a cancer diagnosis (non-cancer controls). In total, cfDNA samples from 534 participants with hematological cancers were included for performance evaluation.

In this second example implementation, the cancer classifier is trained to distinguish between seven hematological subtypes and an absence of cancer (“non-cancer”). The seven hematological subtypes are myeloid neoplasm, non-Hodgkin lymphoma (NHL), circulating lymphoma, plasma cell neoplasm, Hodgkin lymphoma (HL), heme_1, and heme_3. The subtypes heme_1 and heme_3 refer to two types of hematological precursor conditions that may develop into hematological cancers such as the other hematological subtypes. Hematological precursor conditions may include, but are not limited to, monoclonal gammopathy of uncertain significance or monoclonal B cell lymphocytosis. A cross-validated mutual information-based algorithm was used to identify features that discriminated between the seven hematological subtypes and the non-cancer class. A multinomial classifier was then trained to detect the presence or absence of cancer and predict tissue of origin among the five hematological cancers and non-cancers using 6-fold cross-validation.

FIG. 19 illustrates a graph plotting the anomaly scores of a plurality of training samples for hematological-specific cancer classification. In reference to the graph, “M” refers to the myeloid neoplasm hematological subtype, “H3” refers to the heme_3 hematological subtype, “HL” refers to the Hodgkin lymphoma hematological subtype, “nHL” refers to the non-Hodgkin lymphoma subtype, “CL” refers to the circulating lymphoma hematological subtype, “H1” refers to the heme_1 hematological subtype, and “P” refers to the plasma cell neoplasm hematological subtype. On the y-axis are several top features selected for use according to mutual information gain. As such, the first column shows each hematological subtype compared against each of the other hematological subtypes shown in the second column. Across the x-axis are training samples grouped by known hematological subtypes. For example, under column “nHL” are training samples known to be labeled the non-Hodgkin lymphoma hematological subtype. The analytics system determines an anomaly score for each of the selected features. In this implementation, the anomaly score is a binary score based on presence (shown in white) or absence (shown in grey) of an anomalously methylated fragment that covers the feature. Of note, the white regions along the main diagonal (from top left to bottom right) provides an indication to the discriminatory power in classifying the hematological subtypes. Within a row, if a feature is white spread across different samples from different hematological subtypes, there is an indication that the feature has less discriminatory power and is noisy.

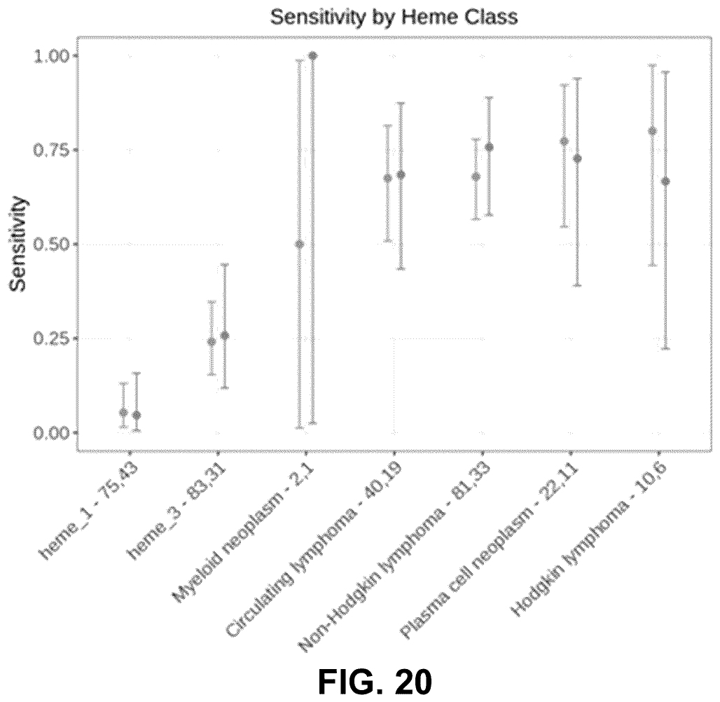

FIG. 20 illustrates a graph showing the hematological-specific cancer classifier's sensitivity at 99.5% specificity. The left set of data for each hematological subtype is for the training set used to train the hematological-specific cancer classifier; whereas, the right set of data is for the holdout set. The number of samples present in the training set and holdout set, respectively, are notated after the label for each hematological subtype across the bottom x-axis. 95% confidence intervals are shown for the sensitivities measured under the training set and the holdout set for each subtype. The heme_1 subtype had a low sensitivity for both the training set and the holdout set. The heme_3 subtype had ˜25% sensitivity for both the training set and the holdout set. The myeloid neoplasm subtype had 50% sensitivity for the training set (accurately predicted 1 in 2 training samples) and 100% sensitivity for the holdout set (accurately predicted 1 in 1 holdout sample). The circulating lymphoma subtype had ˜70% sensitivity for both sets. The non-Hodgkin lymphoma subtype had ˜70% sensitivity for the training set and ˜75% sensitivity for the holdout set. The plasma cell neoplasm subtype had ˜75% sensitivity for both sets. The Hodgkin lymphoma subtype had 80% for the training set and ˜70% for the holdout set.

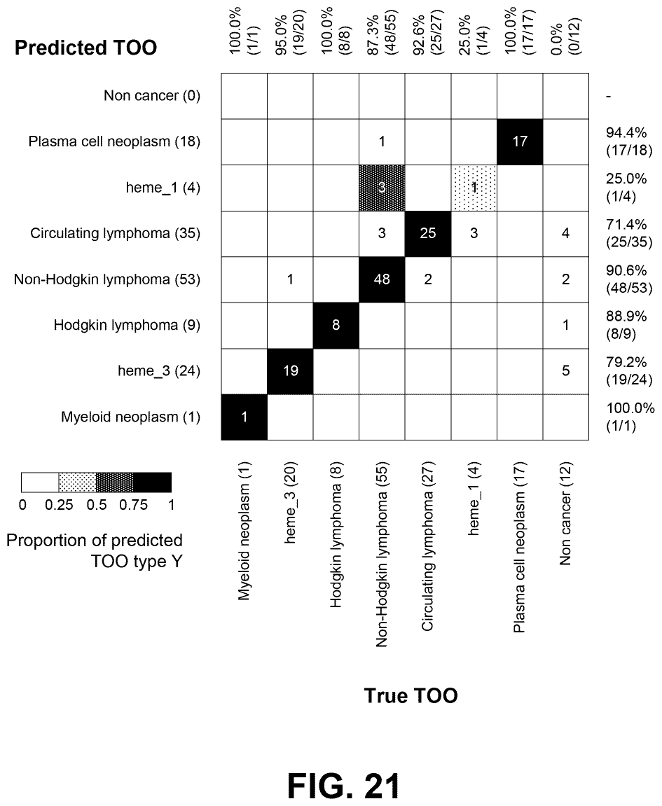

FIG. 21 illustrates a confusion matrix showing cancer prediction accuracy of the hematologic-specific cancer classifier in the second example implementation. The numbers in each box represent the total number of samples predicted. In the confusion matrix, coloring/shading corresponds to the proportion of the predicted hematological subtype, as indicated to the right of the plot. Likewise, the percentage of predictions that are correct is indicated to the right of the graph. The tissue of origin localization was assessed on cancer cases that were correctly detected by a TOO multiclass classifier as hematological cancers. As shown at FIG. 21, the hematological-specific classifier achieved an overall TOO prediction accuracy of ˜75%. Plasma cell neoplasm subtype had a prediction accuracy of 100% with 17 out of 17 known samples accurately predicted. Heme_1 subtype had a prediction accuracy of 25% with 1 out of 4 known samples accurately predicted. Circulating lymphoma subtype had a prediction accuracy of 92.6% with 25 out of 27 known samples accurately predicted. Non-Hodgkin lymphoma subtype had a prediction accuracy of 87.3% with 48 out of 55 known samples accurately predicted. Hodgkin lymphoma subtype had a prediction accuracy of 100% with 8 out of 8 known samples accurately predicted. Heme_3 subtype had a prediction accuracy of 95% with 19 out of 20 known samples accurately predicted. Myeloid neoplasm had a prediction accuracy of 100% with 1 out of 1 known sample accurately predicted.

As demonstrated by the second example hematological-specific classifier, the custom classifier for hematological malignancies and hematological precursor conditions is also capable of identifying such precursor conditions that can eventually devolve into hematological malignancies. This classification capability of precursor conditions proves helpful in identifying individuals that might later develop hematological malignancies, which can lead to even earlier clinical diagnosis and treatment selection. In this way, the custom classifier can achieve even more sensitive detection of multiple cancers and may be used to refine cancer detection and TOO prediction accuracy.

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

What is claimed is:

1. A method for determining a change in a cancer presence of a subject of cancer in a test sample, the method comprising:
responsive to treating a subject with a cancer treatment based on determining an initial cancer presence at an initial tissue of origin (“TOO”) using a cancer detection model, accessing a test sample physically obtained from the subject comprising cell-free DNA (“cfDNA”) test sequences from a sequencing assay, the cell-free DNA test sequences comprising sequencing data representing cancer presence and tissue of origin;
applying a classification model to the test sample to classify the test sample with a cancer score and a tissue signal for a tissue label representing a TOO of a plurality of TOOs, the classification model configured to predict TOO, tissue signal, and cancer score based on sequencing information for the test sample;
selecting a stratum of a plurality of strata based on the tissue signal for the tissue label, the plurality of strata comprising a high tissue signal stratum for the tissue label and a low tissue signal stratum for the tissue label, each stratum of the plurality of strata used to detect at least one different cancer type associated with the tissue label;
applying the cancer detection model comprising a machine-learned model to the test sample, the cancer detection model determining whether the test sample is associated with a subsequent cancer presence or cancer absence in the TOO by comparing the cancer score of the test sample against a binary threshold cutoff for the stratum selected based on the tissue signal for the tissue label; and
determining a change in the cancer presence of the subject due to the cancer treatment based on (1) the initial cancer presence in the TOO determined by the cancer detection model, (2) an initial stratum selected for the initial cancer presence, (3) the subsequent cancer presence or cancer absence in the TOO determined by the cancer detection model, and (4) the stratum selected when determining the subsequent presence or absence of cancer.

2. The method of claim 1, wherein the test sample comprises a test feature vector representing methylation states of the test sample and determined according to methylation information in the sequencing data of the test sample.

3. The method of claim 2, wherein the cancer score is determined by applying a binary cancer classifier to the test feature vector.

4. The method of claim 2, wherein the tissue signal for the TOO is a TOO prediction determined by applying a multiclass cancer classifier to the test feature vector.

5. The method of claim 4, wherein the TOO prediction comprises a prediction value for each of a plurality of tissue labels, each tissue label representing a TOO of the plurality of TOOs, and each prediction value indicating a likelihood that the test sample corresponds to a cancer type associated with the tissue label.

6. The method of claim 5, wherein selecting the stratum of the plurality of strata based on the tissue signal for the tissue label comprises:
determining whether the tissue signal for the tissue label is at or above a prediction value threshold;
responsive to determining that the tissue signal for the tissue label is at or above the prediction value threshold, selecting the high tissue signal stratum; and
responsive to determining that the tissue signal for the tissue label is below the prediction value threshold, selecting the low tissue signal stratum.

7. The method of claim 5, wherein the TOO prediction comprises one or more top predictions, each of the one or more top predictions corresponding to a tissue label of a plurality of tissue labels, wherein a top prediction indicates that the test sample is predicted to have a cancer type associated with the tissue label of the top prediction.

8. The method of claim 7, wherein selecting the stratum of the plurality of strata comprises:
determining whether the tissue label corresponds to a first top prediction of the one or more top predictions;
responsive to determining that the tissue label corresponds to the first top prediction, selecting the high tissue signal stratum; and
responsive to determining that the tissue label does not correspond to the one or more top predictions, selecting the low tissue signal stratum.

9. The method of claim 8, wherein selecting the stratum of the plurality of strata comprises:
determining whether the tissue label corresponds to a second top prediction of the one or more top predictions;
responsive to determining that the tissue label corresponds to the second top prediction, selecting the high tissue signal stratum; and
responsive to determining that the tissue label does not correspond to the one or more top predictions, selecting the low tissue signal stratum.

10. The method of claim 1, wherein the plurality of strata includes a medium tissue signal strata for the tissue label.

11. The method of claim 1, wherein the test sample has an additional tissue signal for an additional tissue label representing an additional TOO of the plurality of TOOs, wherein selecting a stratum of a plurality of strata is further based on the additional tissue signal for the additional tissue label.

12. The method of claim 1, wherein a binary threshold cutoff for each stratum in the plurality of strata is determined by:
obtaining a holdout set of samples comprising cell-free DNA test sequences from a population sequencing assay, each sample in the holdout set of samples having a holdout cancer score and a holdout tissue signal for the tissue label;
stratifying the holdout set of samples into the plurality of strata based on the holdout tissue signals for the tissue label; and
for each stratum of the plurality of strata:
sweeping through a domain of cancer scores at a plurality of candidate binary threshold cutoffs by calculating a true positive rate and a false positive rate for each candidate binary threshold cutoff based on the holdout cancer scores of the samples in the stratum, and
selecting a binary threshold cutoff from the plurality of candidate binary threshold cutoffs for the stratum based on a false positive budget for the stratum and the calculated false positive rates.

13. The method of claim 1, wherein:
the test sample comprises at least 10,000 cfDNA test sequences,
applying the classification model to the test sample comprises applying the classification model to the at least 10,000 cfDNA test sequences, and
applying the cancer detection model to the test sample comprises applying the cancer detection model to the at least 10,000 cfDNA test sequences.

14. The method of claim 1, wherein:
the test sample comprises at least 100,000 cfDNA test sequences,
applying the classification model to the test sample comprises applying the classification model to the at least 100,000 cfDNA test sequences, and
applying the cancer detection model to the test sample comprises applying the cancer detection model to the at least 100,000 cfDNA test sequences.

15. The method of claim 1, further comprising:
generating at least 500 feature vectors for cfDNA fragments in the cfDNA test sequences of test sample, each feature vector representing methylation states of sites in the cfDNA fragments, and
wherein:
applying the classification model to the test sample comprises applying the classification model to the at least 500 feature vectors, and
applying the cancer detection model to the test sample comprises applying the cancer detection model to the at least 500 feature vectors.

16. The method of claim 1, further comprising:
generating at least 2000 feature vectors for cfDNA fragments in the cfDNA test sequences of the test sample, each feature vector representing methylation states of sites in the cfDNA fragments, and
wherein:
applying the classification model to the test sample comprises applying the classification model to the at least 2000 feature vectors, and
applying the cancer detection model to the sample comprises applying the cancer detection model to the at least 2000 feature vectors.

17. The method of claim 1, further comprising:
determining whether the cancer treatment was successful based on the change in cancer presence of the test subject.

18. A system for determining a change in a cancer presence of a subject in a test sample, the system comprising:
a computer processor; and
a non-transitory computer-readable storage medium storing instructions that, when executed by the computer processor, cause the computer processor to perform operations comprising:
responsive to treating a subject with a cancer treatment based on determining an initial cancer presence at an initial tissue of origin (“TOO”) using a cancer detection model, accessing a test sample physically obtained from the subject comprising cell-free DNA (“cfDNA”) test sequences from a sequencing assay, the cell-free DNA test sequences comprising sequencing data representing cancer presence and tissue of origin;
applying a classification model to the test sample to classify the test sample with a cancer score and a tissue signal for a tissue label representing a TOO of a plurality of TOOs, the classification model configured to predict TOO, tissue signal, and cancer score based on sequencing information for the test sample;
selecting a stratum of a plurality of strata based on the tissue signal for the tissue label, the plurality of strata comprising a high tissue signal stratum for the tissue label and a low tissue signal stratum for the tissue label, each stratum of the plurality of strata used to detect at least one different cancer type associated with the tissue label;
applying the cancer detection model comprising a machine-learned model to the test sample, the cancer detection model determining whether the test sample is associated with a subsequent cancer presence or cancer absence in the TOO by comparing the cancer score of the test sample against a binary threshold cutoff for the stratum selected based on the tissue signal for the tissue label; and
determining a change in the cancer presence of the subject due to the cancer treatment based on (1) the initial cancer presence in the TOO determined by the cancer detection model, (2) an initial stratum selected for the initial cancer presence, (3) the subsequent cancer presence or cancer absence in the TOO determined by the cancer detection model, and (4) the stratum selected when determining the subsequent presence or absence of cancer.
